(19) United States
(12) Patent Application Publication
(10) Pub. No.: US 2025/0318819 A1
(43) Pub. Date: Oct. 16, 2025

(54) TISSUE SCAFFOLDING OCCLUSION DEVICE

(71) Applicant: RECROSS CARDIO INC., Dover, DE (US)

(72) Inventors: Thomas Gerhardt, Cape Town (ZA); Boaz Harari, Ganei Tikva (IL); Khusheel Vallabh, Cape Town (ZA); Phillipp Frohmann, Western Cape (ZA); Brandon Reabow, East London (ZA)

(21) Appl. No.: 19/238,198

(22) Filed: Jun. 13, 2025

Related U.S. Application Data
(63) Continuation of application No. PCT/US2023/084087, filed on Dec. 14, 2023.
(60) Provisional application No. 63/387,588, filed on Dec. 15, 2022.
(60) Provisional application No. 63/493,965, filed on Apr. 3, 2023.

(51) Int. Cl. A61B 17/00

(52) U.S. Cl. CPC A61B 17/0057; A61B 2017/00592; A61B 2017/00606

(57) ABSTRACT

According to some embodiments, an interseptal occluding device comprises a support structure comprising a first anchoring portion and an opposite second anchoring portion, a lumen extending through a center of the first anchoring portion and a center second anchoring portion, wherein the support structure is configured to contract and expand between a compressed tubular configuration for insertion through a patient's vasculature, and an expanded configuration, in which the first and second anchoring portions extend radially outwards from the lumen; and a membrane coupled to the first anchoring portion, the membrane configured to occlude a majority of the lumen when the support structure is expanded, the membrane configured to promote tissue growth at least across the membrane.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit of U.S. Provisional Patent Application No. 63/493,965, filed Apr. 3, 2023, and U.S. Provisional Patent Application No. 63/387,588, filed Dec. 15, 2022, the entire contents of each of these applications are hereby incorporated by reference herein in their entirety.

### BACKGROUND

### Field

This application relates generally to medical devices, and more specifically, in certain arrangements to apparatuses, systems, and method for occluding interatrial partition holes/openings.

### Description of the Related Art

In a heart, an atrial septum separates a right atrium from a left atrium and a ventricular septum separates the left ventricle from the right ventricle. Defects (e.g., holes or openings) in the septum can occur congenitally or by piercing the septum with a medical instrument to access a position within the heart. In addition, non-congenital defects such as patent foramen ovale can persist into adulthood and are common. Implantable medical devices exist for treating a number of diseases and conditions associated with the heart. For example, occlusive devices can be used to obstruct (e.g., completely, or partially) the flow of blood through a defect in a ventricular or atrial septum.

The femoral vein is an access point for many laboratory catheterization procedures, with a smaller percentage of procedures using the access to arteries. The atrial partition is a percutaneous access point, for example for atrial fibrillation therapy, closure of the left atrial appendage, percutaneous repair of the mitral valve, and percutaneous replacement of the mitral valve. In these and other procedures, the devices need to cross the atrial partition and, in doing so, can leave an orifice in the atrial partition which cannot close or heal on its own.

### SUMMARY

The chronic and debilitating effects of septal defects affect millions of people around the world. An estimated 4.2 million people are born with an atrial septal defects (ASD) every year and an estimated 27% of people are born with persistent patent foramen ovale (PFO). Studies have shown that 40-60% of ASD patients develop atrial fibrillation (AF) and approximately 20% of ASD patients develop mitral regurgitation. Living with an ASD is a burden and often results in shortness of breath, extreme fatigue, peripheral edema, heart palpitations, and an increased risk of stroke. ASDs can cause chromic atrial stretching and consequent atrial arrythmias (AA). Studies have also shown that 40% of cryptogenic stroke is linked to PFO. PFO has serious implications including an increased risk of stroke and required migraine treatment.

Current treatment of ASD and PFO by permanent septal occlusive devices is often used as a last resort and can result in significant complications. Some physicians recommend AF treatment prior to closure due to the restricted left atrium access once an occlusive device is implanted. Currently, only symptomatic defects are closed or those with suspicion of paradoxical embolism. Current occlusive devices for treating septal defects also have a number of issues. For example, current devices often include a protruding, bulky mesh component which can induce chronic inflammation, a slow healing response, and increase the risk of stroke. Current devices often include stiff and dense braids, which can disrupt the conduction network, increase the risk of arrythmias, result in loss of septal compliance, and may increase the risk of erosions within the septum. Once implanted, current devices have poor elasticity and low compliance in part due to the use of PTFE, which can cause acute thrombus formation and intimal hyperplasia. Further, patients are often required to undergo long-term medical therapy including Dual Antiplatelet Therapy (DAPT) & Aspirin/Clopidogrel for three-six months and up to five years after device placement. The long-term medical therapy is a burden for patients and can result in complicated side effects. Additionally, once implanted, these devices obstruct septal access and limit or remove future treatment options. While maintaining left atrium access is crucial for all patients, it is specifically important for congenital heart disease (CHD) patients who suffer from high rates of cardiac comorbidities. Approximately 50% of CHD patients develop atrial fibrillation by age 65, which results in a significant increase in the risks of stroke, heart failure, congenital cardiac intervention, arrythmia intervention, and non-congenital cardiac intervention.

Various systems, methods, and devices are disclosed herein for treatment of septal defects, including ostium secundum ASDs, by providing interseptal occlusion (e.g., to block blood flow between the right and left atriums of a heart) while continuing to allow for future septal access and reducing the negative side effects of an occlusion procedure. The systems, methods, and devices of the disclosure each have several innovative aspects, no single one of which is solely responsible for the desirable attributes disclosed herein.

The occlusion devices described herein may include an electrospun microfiber or nanofiber material membrane, which demonstrates substantially superior endothelial cell growth when compared to available ePTFE & woven PET. Electrospun PU nanofiber scaffolds support the formation of stable endothelial cell monolayers similar to vascular endothelium and cardiac muscle. Formation of smooth endothelium may reduce the shear forces to prevent thrombus formation. Elastic nanofiber material is also associated with greatly reduced inflammatory response compared to non-compliant alternatives such as PTFE/PET. The occlusion devices described herein may comply with the complex cardiac structures of the heart by septal compliance and contractility. Once implanted, the occlusion devices promote the growth of a biostable cell scaffold via rapid tissue regeneration, often resulting in a full tissue layer grown within eight weeks.

The occlusion devices described herein enable transseptal access for a multitude of therapeutic options post implantation and may reduce/abolish the need for DAPT following a procedure. Once implanted, the non-cell porous structure results in immediate septal occlusion and the elastic membrane mimics septal compliance. The occlusion devices may include a lightweight frame that allows for compliant anchorage which protects the surrounding tissue. Further, a smooth left atrium membrane reduces the stroke risk for patients once implanted.

The occlusion devices described herein may be constructed from extremely lightweight materials. As a result of the lightweight materials, the septum is able to better preserve its compliant nature. Septal compliance is important from a hemodynamic perspective, as it more closely mimics the natural movement of cardiac tissue. Furthermore, a more flexible, conformable device better protects complex cardiac anatomies both within the septum (such as conduction fibres) and adjacent to the septum (such as the aorta). Lastly, a lightweight device is innately desirable as the less material implanted into the body, the lower the immune response. The best device is less device. Additionally, some patients have a hypersensitivity to nickel which would be reduced with a lower mass of nitinol implanted.

According to some embodiments, an interseptal occluding device comprising a support structure comprising a first anchoring portion and an opposite second anchoring portion, a lumen extending through a center of the first anchoring portion and a center second anchoring portion, wherein the support structure is configured to contract and expand between a compressed tubular configuration for insertion through a patient's vasculature, and an expanded configuration, in which the first and second anchoring portions extend radially outwards from the lumen; and a membrane coupled to the first anchoring portion, the membrane configured to occlude a majority of the lumen when the support structure is expanded, the membrane configured to promote tissue growth at least across the membrane.

According to some embodiments, a method, comprises inserting a delivery system into a patient's vasculature, the delivery system comprising a release member and a sheath, the sheath containing an interseptal occluding device in a compressed configuration, the interseptal occluding device comprising a first anchoring portion and an opposite second anchoring portion, the second anchoring portion coupled to the release member; advancing a distal end of the sheath at least partially across a partition in the patient's heart; advancing the interseptal occluding device within the sheath such that the first anchoring portion expands in a first compartment on a first side of the partition; advancing the interseptal occluding device outside of the sheath such that the second anchoring portion expands in a second compartment on a second side of the partition, the second side opposite the first side, wherein the second anchoring portion remains coupled to the release member in an expanded configuration; and releasing the interseptal occluding device from the delivery system.

According to some embodiments, an interseptal occluding device configured to be implanted in a heart of a patient comprises a support structure comprising a lumen; and a membrane comprising a plurality of electrospun fibers, the membrane coupled to at least a portion of the support structure, the membrane configured to occlude a majority of the lumen when the interseptal occluding device is implanted.

According to some embodiments, an interseptal occluding device configured to be implanted in a heart of a patient comprises a support structure comprising a central structure; and a membrane configured to promote tissue growth across the central structure, the membrane coupled to at least a portion of the support structure, the membrane configured to occlude a majority of the central structure when the interseptal occluding device is implanted.

According to some embodiments, an interseptal occluding device comprises a support structure comprising a central structure, a first anchoring portion and an opposite second anchoring portion; and a membrane coupled to the first anchoring portion, the membrane configured to occlude a majority of the central structure when the support structure is expanded, the membrane configured to promote tissue growth at least across the membrane.

### BRIEF DESCRIPTION OF THE DRAWINGS

These and other features, aspects and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 illustrates a sectional view of a schematic of a heart in which defects or openings are present in the atrial and ventricular partitions;

FIG. 2A illustrates a perspective view of an occluding device according to one embodiment;

FIG. 2B1 illustrates a left atrial side view of the occluding device of FIG. 2A including an occlusion membrane with slits;

FIG. 2B2 illustrates a left atrial side view of the occluding device of FIG. 2A including an electrospun occlusion membrane;

FIG. 2C illustrates a right atrial side view of the occluding device of FIG. 2A;

FIG. 2D illustrates a side view of the occluding device of FIG. 2A;

FIG. 2E illustrates a section view of the occluding device of FIG. 2A along the line 2E-2E in FIG. 2B1;

FIG. 3A illustrates a perspective view of an occluding device frame according to one embodiment;

FIG. 3B illustrates a left atrial side view of the occluding device frame of FIG. 3A;

FIG. 3C illustrates a right atrial side view of the occluding device frame of FIG. 3A;

FIG. 3D illustrates a side view of the occluding device frame of FIG. 3A;

FIG. 4A illustrates a sectional view of a schematic of a heart partition in a pre-operative state;

FIG. 4B illustrates a sectional view of a schematic of a heart partition in an intra-operative state;

FIG. 4C illustrates a sectional view of a schematic of a heart partition in a post-operative state;

FIG. 5A illustrates a delivery system for an occluding device according to one embodiment;

FIG. 5B-5C illustrate a device loading component of the delivery system of FIG. 5A;

FIG. 6A illustrates a side view of the grasping system according to one embodiment;

FIG. 6B illustrates a perspective view of the grasping system of FIG. 6A;

FIG. 7A-7D illustrate the grasping system of FIG. 6A;

FIG. 8A-8E illustrate an example deployment of an occluding device according to an embodiment;

FIG. 9A illustrates a perspective view of an occluding device according to one embodiment;

FIG. 9B illustrates a left atrial side view of the occluding device of FIG. 9A;

FIG. 9C illustrates a right atrial side view of the occluding device of FIG. 9A;

FIG. 9D illustrates a side view of the occluding device of FIG. 9A;

FIG. 9E illustrates a section view of the occluding device of FIG. 9A;

FIG. 10A illustrates a perspective view of an occluding device frame according to one embodiment;

FIG. 10B illustrates a left atrial side view of the occluding device frame of FIG. 10A;

FIG. 10C illustrates a right atrial side view of the occluding device frame of FIG. 10A;

FIG. 10D illustrates a side view of the occluding device frame of FIG. 10A;

FIG. 10E illustrates a side view of an occluding device frame;

FIG. 10F illustrates a detailed view of an attachment portion of the occluding device frame of FIG. 10E;

FIGS. 10G and 10H illustrate detailed views of a portion of the occluding device frame of FIG. 10E coupled to a portion of the delivery system of FIG. 5A;

FIG. 11A illustrates a left atrial side view of an occluding device;

FIG. 11B illustrates a right atrial side view of the occluding device of FIG. 11A;

FIG. 11C illustrates a side view of the occluding device of FIG. 11A implanted in a partition of a heart;

FIG. 11D illustrates a side view of the frame of the occluding device of FIG. 11A;

FIG. 11E illustrates a side view of the occluding device of FIG. 11A coupled to a delivery system in an expanded configuration;

FIG. 11F illustrates a schematic side view of a central portion of the occluding device of FIG. 11A;

FIG. 12A illustrates a partial view of a distal end of a delivery system;

FIG. 12B illustrates a section view of the distal end of the delivery system of FIG. 12A;

FIG. 12C illustrates a grasping system of the delivery system of FIG. 12A;

FIG. 13A illustrates a side view of an occluding device frame; and

FIG. 13B illustrates a detailed view of an attachment portion of the occluding device frame of FIG. 13A;

### DETAILED DESCRIPTION OF EMBODIMENTS

Embodiments of the disclosure will now be described with reference to the accompanying figures. The terminology used in the description presented herein is not intended to be interpreted in any limited or restrictive manner, simply because it is being utilized in conjunction with a detailed description of certain embodiments of the disclosure. Furthermore, embodiments of the disclosure may include several novel features, no single one of which is solely responsible for its desirable attributes, or which is essential to practicing the embodiments of the disclosure herein described. For purposes of this disclosure, certain aspects, advantages, and novel features of various embodiments are described herein. It is to be understood that not necessarily all such advantages may be achieved in accordance with any particular embodiment. Thus, for example, those skilled in the art will recognize that one embodiment may be carried out in a manner that achieves one advantage or group of advantages as taught herein without necessarily achieving other advantages as may be taught or suggested herein.

Although the various embodiments disclosed herein have specific relevance to interseptal occlusive devices (e.g., to block blood flow between the right and left atriums of a heart) the features, advantages and other characteristics disclosed herein may have direct or indirect applicability in other applications, such as, for example, systems, methods, and devices for hernia repair, vascular closure, and the like, other types of medical devices, other mechanical devices, and/or the like.

### I. Overview

A partition is for example a thin wall dividing a cavity into two smaller cavities or chambers or compartments. A “partition”, as the term is used herein, is intended to define both a heart wall which divides two atria, as well as a wall which divides the right or left atrium and ventricle.

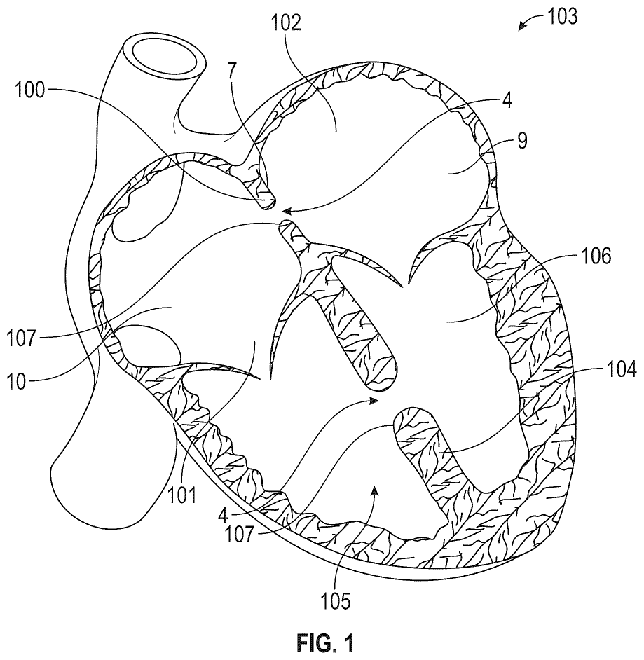

FIG. 1 illustrates a sectional view of a schematic of a heart 103 in which defects or openings are present in the atrial and ventricular partitions. In the heart 103, an atrial partition 100 comprising a tissue wall separates a right atrium 101 from a left atrium 102 and a ventricular partition 104 comprising a tissue wall separates a right ventricle 105 and a left ventricle 106 of the heart 103.

The heart 103 may contain one or more defects. “Defects”, as the term is used herein, may refer to congenital defects and/or interatrial partition hole/openings created following, for example, percutaneous interventions with trans-septal puncture techniques (e.g., for the mitral valve repair, the occlusion of the Left Atrial Appendage, and/or the like). As shown, the heart 103 includes a defect 107 located in the atrial partition 100 and a defect 107 located in the ventricular partition 104. The defect(s) 107 may have been created by piercing the atrial partition 100 or the ventricular partition 104 with a medical instrument to access a position within the heart.

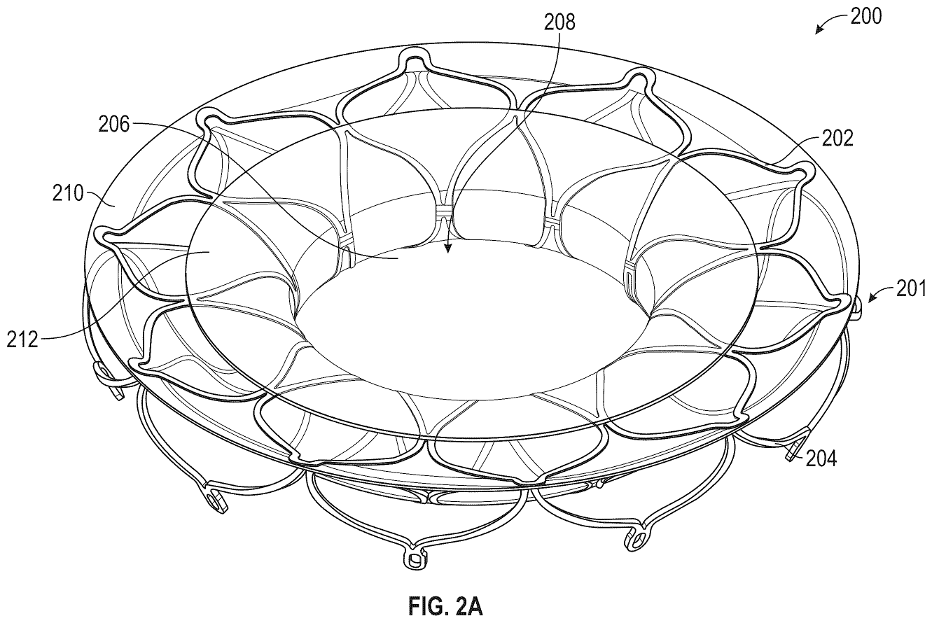

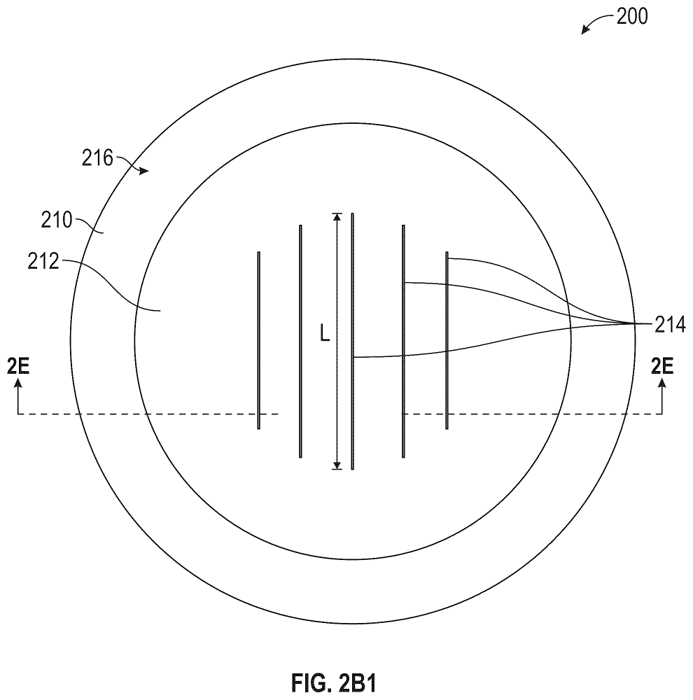

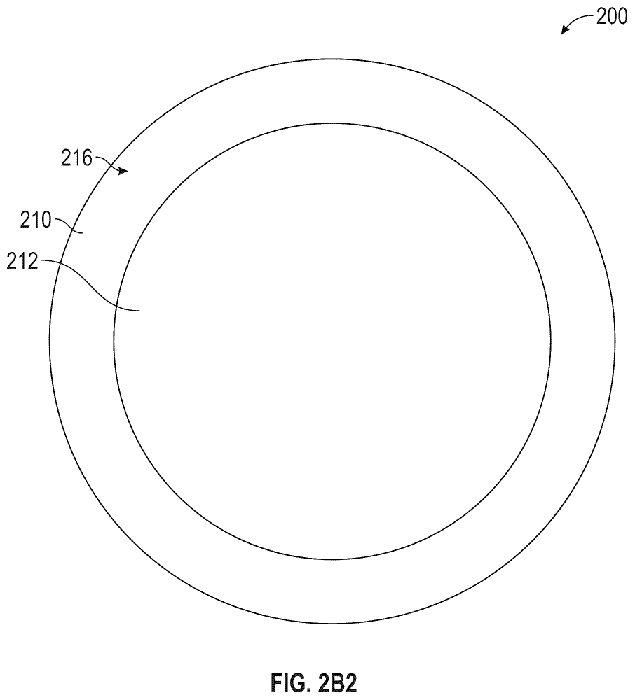

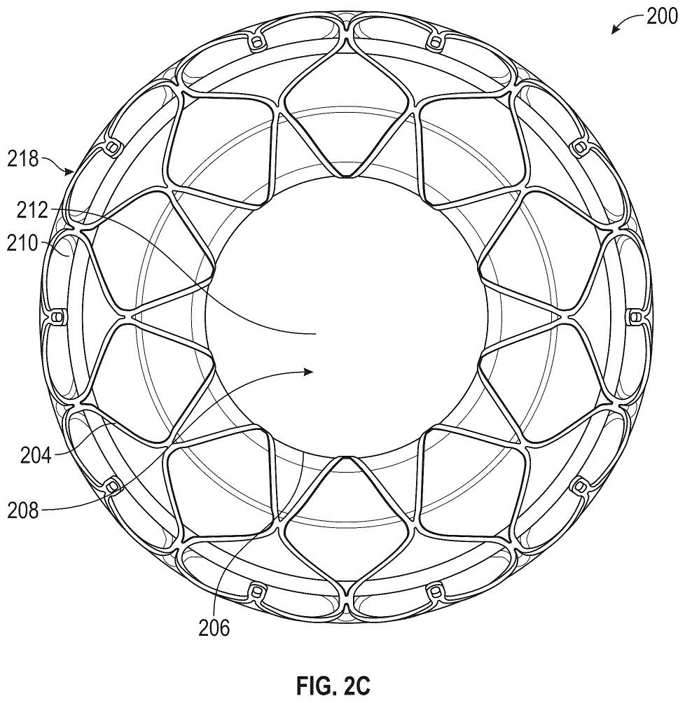

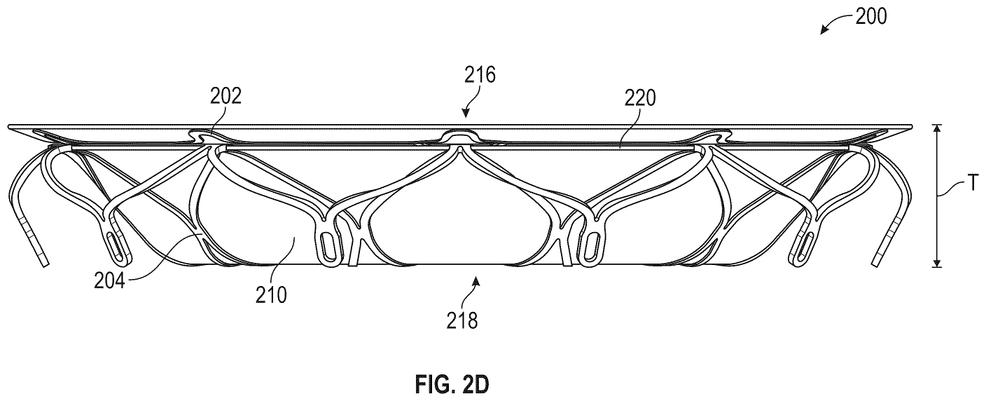

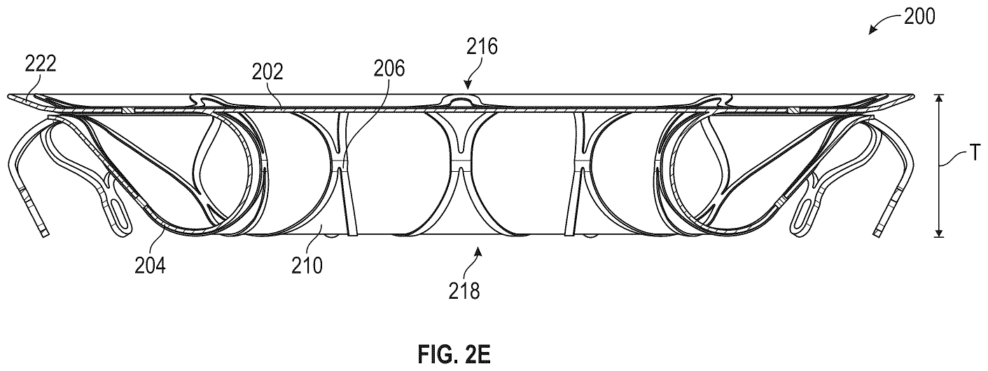

The femoral vein is an access point for many laboratory catheterization procedures, with a smaller percentage of procedures using the access to arteries. The atrial partition 100 is a percutaneous access point, for example for atrial fibrillation therapy, closure of the left atrial appendage, percutaneous repair of the mitral valve, and percutaneous replacement of the mitral valve. In these and other procedures, the devices need to cross the atrial partition 100 and, in doing so, can leave an orifice in the atrial partition which cannot close or heal on its own. In some embodiments, an occluding device, such as, for example, the device illustrated in FIGS. 2A-2E can be implanted in the defect 107 to partially or completely occlude the defect 107.

### II. Occluding Device

a. Overview

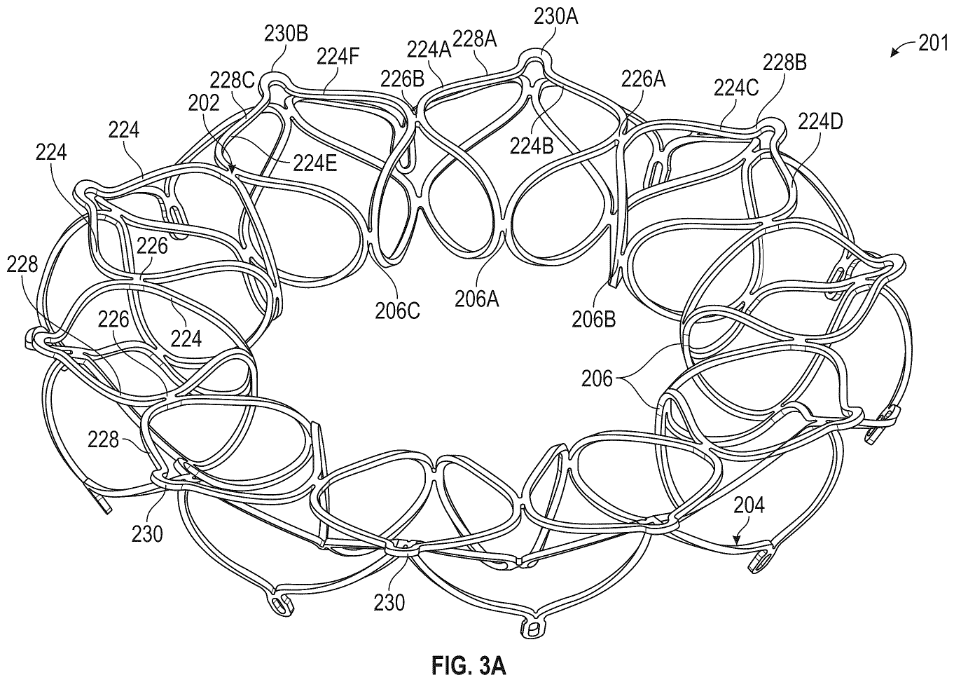

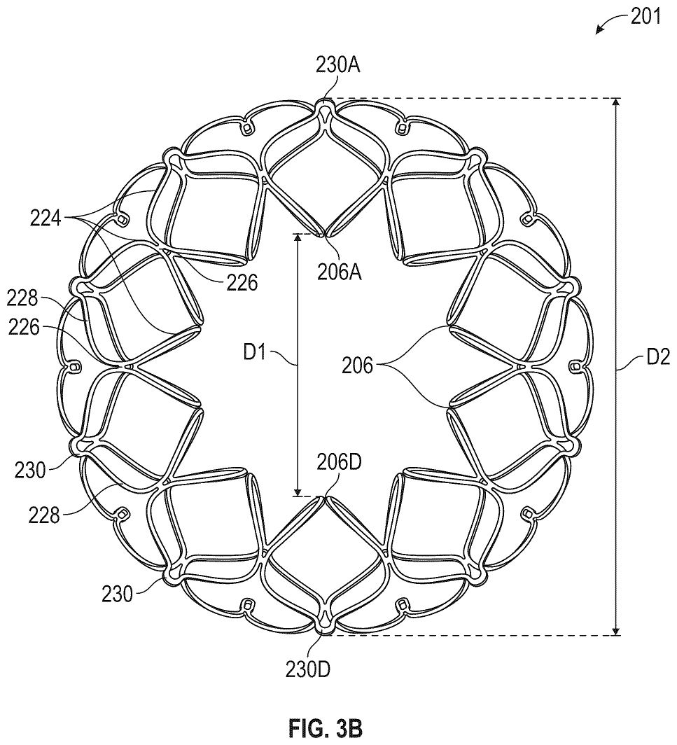

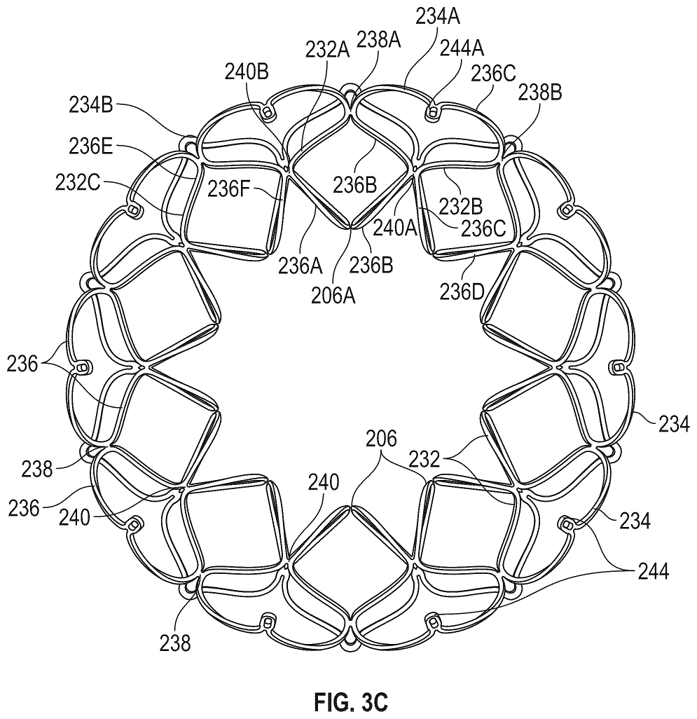

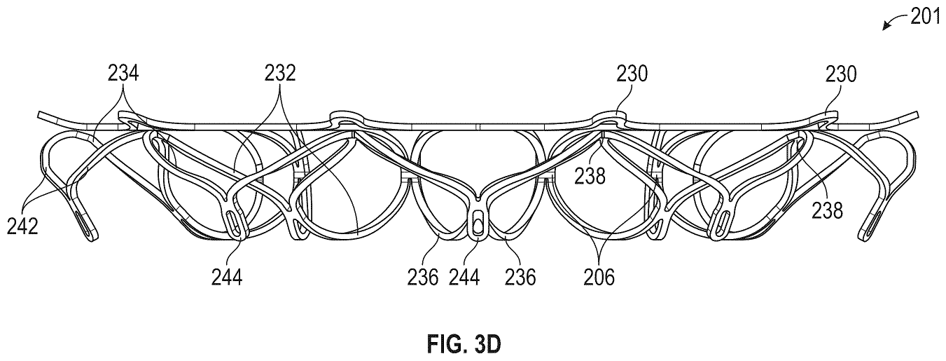

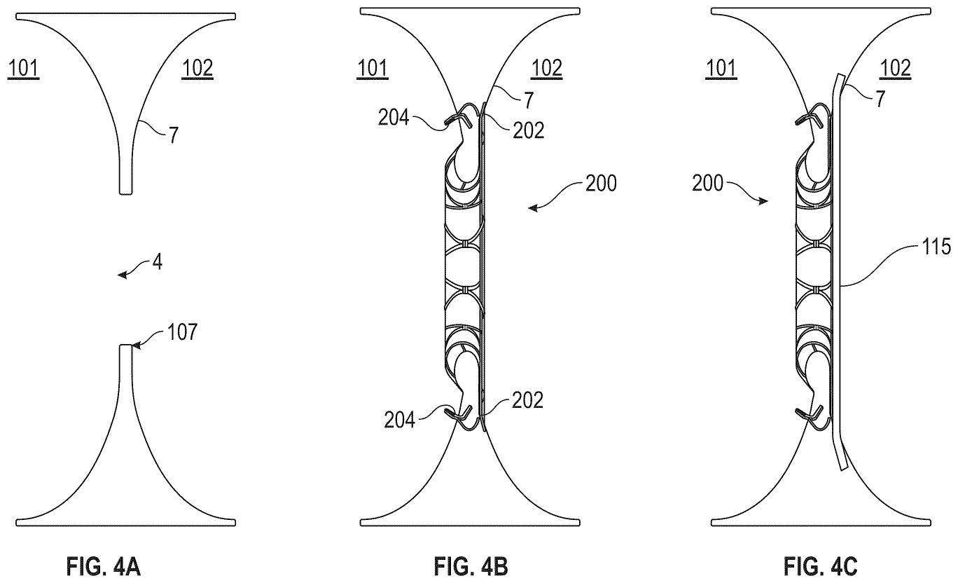

FIGS. 2A-2E illustrate an occluding device 200 in an expanded configuration. With reference to FIG. 2A, the occluding device 200 comprises a support structure 201. The support structure 201 may comprise a first anchoring portion 202 and an opposite second anchoring portion 204. The support structure 201 is configured to expand and contract between a compressed tubular configuration for insertion through a patient's vasculature and an expanded or extended configuration in which the first and second anchoring portions 202, 204, extend radially outwards from a central portion 206. The central portion 206 may define a lumen 208, where the lumen 208 extends through the center of the first anchoring portion 202 and the center of the second anchoring portion 204. As shown in FIGS. 4A-4C, the first and second anchoring portions 202, 204 may be used to compress a partition (e.g., partition 7 of FIG. 1) therebetween. For example, as described further herein, the occluding device 200 may be inserted in a heart 103 to partially or completely occlude a defect 107. For example, where the defect 107 is present in the atrial partition 100, the occluding device 200 may be positioned such that either the first or second anchoring portion 202/204 is positioned on one side of the partition 7 in a first compartment 9 (e.g., corresponding to the right atrium 101) and the opposite anchoring portion 202, 204 is positioned on the opposite side of the partition 7 in a second compartment 10 (e.g., corresponding to the left atrium 102). The support structure 201 is described further with reference to FIGS. 3A-3D.

The occluding device 200 may further include one or more diaphragms or membranes coupled to the support structure 201. For example, in some embodiments, including the embodiment illustrated, the occluding device 200 comprises a frame covering membrane 210 and an occlusion membrane 212. One or both of the membranes 210/212 may be configured to close the lumen 208 when the occluding device 200 is in an extended configuration. The frame covering membrane 210 may be configured to cover all or a portion the first anchoring portion 202 and/or the second anchoring portion 204. For example, frame covering membrane 210 may extend between the first and second anchoring portions 202, 204 across the central portion 206. The occlusion membrane 212 may be positioned on top of the frame covering membrane 210 and may be configured to occlude a flow of blood. “Occlude”, as the term is used herein, may refer to minimizing or completely avoiding a flow of blood passing through a membrane. For example, when the occluding device 200 is positioned within the heart 103 as described above, the occlusion membrane 212 may occlude a lumen 4 corresponding to a defect 107 and prevent the flow of blood from the first compartment 9 to the second compartment 10 or vice-versa.

The occlusion membrane 212 may comprise a roughly circular shape. However, in some embodiments, the occlusion membrane 212 may comprise any suitable shape (e.g., square, rectangle, polygon, and/or the like) to sufficiently cover a majority of the lumen 208 when the occluding device 200 is in an expanded configuration. The occlusion membrane 212 may be coupled to the covering membrane 210 and/or support structure 201 of the occluding device 200 by any suitable means, including, for example, gluing, heat scaling, lamination, spraying, dipping, electrospinning, stitching, co-molding, crimping, interlocking, a combination of the foregoing, and/or the like. Similarly, the frame covering membrane 210 maybe coupled to the support structure 201 by any suitable means, including, for example, gluing, heat sealing, stitching, co-molding, crimping, interlocking, a combination of the foregoing, and/or the like. In some cases, the support structure 201 may be completely encapsulated/enclosed by the frame covering membrane 210, such that there is no portion of the first anchoring portion 202 or the second anchoring portion 204 is not covered by either the frame covering membrane 210 or another covering portion. In certain embodiments, at least 90% of the surface area of the support structure 201 is encapsulated by the frame covering membrane 210, and in certain embodiments at least 80% of the surface area of the support structure 201 is encapsulated by the frame covering membrane. In certain embodiments, only the outside ends of the support structure 201 is exposed (i.e., not encapsulated by the frame covering membrane). For example, in some embodiments, a portion of the support structure 201 that extends outside of the inner 90% diameter of the occluding device 200 is exposed. In another example, in some embodiments, a portion of the support structure 201 that extends outside of the inner 80% diameter of the occluding device 200 is exposed. In some examples, completely encapsulating or encapsulating a majority of the occluding device 200 in a membrane (e.g., the frame covering membrane 210 and/or the occlusion membrane 212) may provide certain benefits over un-encapsulated devices with exposed metal components, such as device with metal braids. For example, devices with exposed metal can cause damage to adjacent structures in the heart, such as the aorta, as a result of the direct contact between the metal components and the heart tissue, as well as a result of the erosion of the metal over time. In some examples, the occlusion membrane 212 may be coupled to the central portion 206 which delimits the lumen 208. In this example, a majority (e.g., 80%, 90%, etc.) or the entirety of the support structure 201 may be encapsulated by one or both of the frame covering membrane 210 and the occlusion membrane 212. As explained herein, the membranes 210, 212 material provides a scaffold for tissue growth. When the entire occluding device 200 is covered with membranes 210, 212, the occluding device 200 does not include exposed metal, which is desirable because it can be harmful to the patient to have exposed metal in contact with circulating blood. Additionally, the occlusion membrane 212 coupled to the central portion 206 functions as an occlusive element, which allows blood cells to quickly cover and subsequently endothelialize on the occluding device 200. In some implementations, multiple or additional membranes with reduced surface area (e.g., of at least 5% relative to a main frame covering membrane 210) may be placed onto the support structure 201 or the main frame covering membrane 210 against one or more features on the occluding device 200 such as anchoring portions 202, 204 or similar peripheral structures, to produce softer regions within the frame covering membranes 210 and promote atraumatic membrane coverage. For example, certain portions of the support structure 201 may include additional membranes layers.

The frame covering membrane 210 may comprise any suitable material that can expand and contract with the occluding device 200. Generally, the frame covering membrane 210 extends from the top edges 222 (e.g., FIG. 2E) of the first anchoring portion 202, along the struts of the first anchoring portion 202, across the central portion 206, and along the struts of the second anchoring portion 204 to the outer edge 220 (e.g., FIG. 2D) of the second anchoring portion 204. Accordingly, a suitable material for the frame covering membrane 210 advantageously is flexible enough to stretch along the support structure 201 while generally conforming to the shape of the support structure 201. For example, the frame covering membrane 210 may comprise an elastic material such as, for example, a silicone or medical elastomer, polyurethane, polyurethane blend, and/or the like. In another example, the frame covering membrane 210 may comprise a mesh material, such as, for example, polyethylene terephthalate (PET), expanded polytetrafluoroethylene (ePTFE), polytetrafluoroethylene (PTFE), polyvinyl alcohol (PVA), and/or the like. In some embodiments, the frame covering membrane 210 may comprise a biodegradable or bioabsorbable material.

The occlusion membrane 212 may comprise any suitable material that can expand and contract with the occluding device 200 and sufficiently occlude a flow of blood. For example, the occlusion membrane 212 may comprise an elastic material such as, for example, a silicone or medical elastomer, polyurethane, polyurethane blend, and/or the like. In another example, the occlusion membrane 212 may comprise a mesh material, such as, for example, polyethylene terephthalate (PET), expanded polytetrafluoroethylene (ePTFE), polytetrafluoroethylene (PTFE), polyvinyl alcohol (PVA), and/or the like. In some embodiments, the occlusion membrane 212 may comprise a biodegradable or bioabsorbable material.

In some embodiments, including the embodiment illustrated in FIG. 2B2, the occlusion membrane 212 and/or the frame covering membrane 210 comprises an electrospun membrane. Electrospun clastic materials are a subset of clastic materials. For example, the occlusion membrane 212 and/or the frame covering membrane 210 may comprise a continuous micro/nano polymer fiber. Use of an electrospun membrane may result in the membranes 210 and/or 212 having high elasticity and/or being highly compliant. For example, when an electrospun polymer is user for the membranes 210, 212, the membranes 210, 212 may be less likely to fail (e.g., tear) when stretched as compared to dipped/sprayed equivalents. An electrospun membrane may also promote the development and growth of a layer of tissue over the occlusion membrane 212 and/or the occluding device 200 once implanted. For example, the electrospun occlusion membrane 212 and/or frame covering membrane 210 may allow for a thin layer of tissue to endothelialize (e.g., allow for rapid endothelization). For example, electrospun materials as used in embodiments disclosed herein advantageously can be associated with better endothelialization (faster and with less inflammation) than non- porous materials, which (while not being bound or limited to any particular theory) may be a result of the matrix of nanofibers better mimicking natural physiology to encourage healthy cell growth. As the tissue layer grows, the occluding device 200 may provide for complete occlusion of the lumen 4 and may also reduce the risk of thrombus. In an embodiment where tissue growth is promoted, the occluding device 200 may act as a scaffold for tissue growth and serve as a prosthetic septum for the heart 103. Additionally, due in part to the compliant properties of the electrospun membrane (e.g., occlusion membrane 212), there may be less inflammation in the septal tissue when the occluding device 200 is implanted when compared to other non-compliant materials such as PET and PTFE. As noted above, less inflammation may promote rapid endothelization. The electrospun nanofiber membrane may support the formation of stable endothelial cell monolayers similar to vascular endothelium and cardiac muscle. In some embodiments, all or a portion of the frame covering membrane 210 may be configured to minimize tissue growth on the frame covering membrane 210. For example, it may be desirable for a minimal amount of tissue to grow within the central portion 206/the inner boundaries of the lumen 208.

Use of clastic occlusion technology, such as an electrospun membrane, for the occlusion membrane 212 and/or the frame covering membrane 210 may provide a number of important benefits for the occluding device 200, including improved septal compliance. For example, the electrospun membrane may mimic septal compliance. In one example, the endothelialization of the occluding device 200 may be improved due in part to the compliant nature of the occlusion membrane 212 and/or frame covering membrane 210. Compliant materials, such polyurethanes and silicones, demonstrate better healing and lower inflammatory responses when compared to non-compliant inelastic materials such as PET, ePTFE, PTFE, and the like. In another example, the hemodynamics of a patient may improve when a compliant material is used, as a compliant neo-septum better mimics a natural septum, which allows pressures within the heart to better mimic natural physiology. In yet another example, use of a compliant material may improve the response from the structures surrounding the occluding device 200 once implanted compared to non-compliant materials. For example, when a stiff occluder or material (i.e., non-compliant) is used, poor healing often results due to persistent inflammation. As inflammation continue, it may result in stiffening of the septum, which may have significant implications. For example, there may be damage to the electrical conduction system which runs through the entire septum of the heart (i.e., both the atrial and ventricular septums). When the electrical conduction system is damaged, a patient is at an increased risk for atrial arrythmias, which is why occluding devices are often associated with atrial arrythmias.

In some embodiments, the occluding device 200 may comprise one or more frame covering membrane(s) 210. For example, the occluding device 200 may comprise 1, 2, 3, 4, 5, 6, 7, 8, 9, 10, and/or the like frame covering membranes 210. When multiple frame covering membranes 210 are present, the multiple membranes may be layered on top of each other and/or different frame covering membrane 210 may cover different portions of the support structure 201. For example, one or more frame covering membrane(s) 210 may cover the support structure 201, while one or more frame covering membrane(s) 210 cover the second anchoring portion 204. In some embodiments, the occluding device 200 may comprise one or more occlusion membrane(s) 212. For example, the occluding device 200 may comprise 1, 2, 3, 4, 5, 6, 7, 8, 9, 10, and/or the like occlusion membranes 212. When multiple occlusion membranes 212 are present, the multiple membranes may be layered on top of each other.

FIGS. 2B1 and 2B2 illustrate left atrial side views of the occluding device 200 including occlusion membranes 212 according to different embodiments. FIG. 2C illustrates a right atrial side view of the occluding device 200. In some cases, it may be desirable to re-cross the partition 7 after the occluding device 200 is positioned within the heart 103. For example, in some cases, a subsequent medical procedure may require that the same lumen 4 corresponding to a now occluded defect 107 be re-crossed to gain access to the first compartment 9 through the second compartment 10 or vice-versa. In some embodiments, including the embodiments illustrated in FIGS. 2B1 and 2B2, the occluding device 200 may be configured to allow a medical instrument (e.g., a catheter) to pass through the occlusion membrane 212 and the lumen 208 such that an opposite compartment of the heart can be accessed through the occluding device 200. Generally, the occluding device 200 may be re-crossed in a subsequent medical procedure months or years after the occluding device 200 is initially implanted in the heart 103; however, in some embodiments, the occluding device 200 may be re-crossed shortly after implantation. In an embodiment where one or both the frame covering membrane 210 and occlusion membrane 212 are configured to promote tissue growth, recrossing the occluding device 200 may require both the layer of tissue formed on the occlusion membrane 212 (e.g., tissue layer 115 of FIG. 4C) and the occlusion membrane 212 to be punctured to cross through the lumen 208. While the occlusion membrane 212 is shown as being positioned near one side of the occluding device 200, it is recognized that the occlusion membrane 212 can be positioned anywhere within or on the occluding device 200. For example, the occlusion membrane 212 can be positioned at different locations along the neck defined by the central portion 206. In one example, the occlusion membrane 212 may be positioned primarily on the right atrial side or the left atrial side. In another example, the occlusion membrane 212 may be positioned centrally within the neck defined by the central portion 206 between the right and left atrial sides. The position of the occlusion membrane 212 on the occluding device 200 and/or relative to the frame covering membrane 210 can change the location of the eventual tissue layer 115 formed on the occluding device 200. For example, when the occlusion membrane 212 is positioned within the central portion 206, the tissue layer 115 may be formed within the occluding device 200 and within the central portion 206.

In some embodiments, including the embodiment illustrated in FIG. 2B1, the occlusion membrane 212 may comprise a plurality of cuts or slits 214 to facilitate the occluding device 200 to be crossed by a medical instrument. For example, the occlusion membrane 212 may include, 1, 2, 3, 4, 5, 10, 15, 20, 30, 50, and/or the like slits 214. The length L of an individual slit 214 may vary based on the position of the slit 214 on the occlusion membrane 212. In some embodiments, each individual slit 214 may extend a percentage of the length of a chord line when the occlusion membrane 212 has a circular shape. “Chord line”, as the term is used herein, may refer to a line segment joining any two points on the circumference of the circle. For example, an individual slit 214 may have a length extending between 10% and 100% of the chord line (e.g., 10% to 100%, 20% to 90%, 30% to 75%, 50% to 60%, values between the foregoing, etc.). Generally, the slits 214 are roughly parallel to each other. In operation, when a medical instrument crosses the occlusion membrane 212, the medical instrument may pass through one slit 214 of the plurality of slits 214 and the occlusion membrane 212 may elastically deform to allow the medical instrument to pass through the occluding device 200. Once the medical instrument is removed from the occluding device 200 (e.g., retracted back through a patient's vasculature), the occlusion membrane 212 may be configured to return to its original position to continue occluding the lumen 4. In an embodiment where the occluding device 200 comprises multiple occlusion membranes 212, the multiple membranes 212 may be layered such that the slits 214 of a first membrane 212 are perpendicular or at different angles to the slits 214 of a second membrane 214. For example, when the occluding device 200 is positioned within the heart 103, the slit(s) 214 of the first occlusion membrane 212 may run approximately vertically, while the slit(s) 214 of the second occlusion membrane 212 may run approximately horizontally.

In some embodiments, the occlusion membrane 212 may comprise an individual slit 214 which may extend roughly across the center of the occlusion membrane 212. The length L of the individual slit 214 may extend a percentage of the length of a chord line corresponding to the diameter of the occlusion membrane 212 when the occlusion membrane 212 has a circular shape. For example, the individual slit 214 may have a length extending between 10% and 100% of the diameter chord line (e.g., 10% to 100%, 20% to 90%, 30% to 75%, 50% to 60%, values between the foregoing, etc.).

In some embodiments, including the embodiment illustrated in FIG. 2B2, the occlusion membrane 212 comprises an electrospun membrane. In the embodiment of FIG. 2B2, the occlusion membrane 212 may not include any slits 214. Instead, the occlusion membrane 212 may include a plurality of perforations, such as, for example, 1, 5, 10, 25, 50, 100, 500, 1000, 10,000, 100,000, and/or the like perforations. An electrospun occlusion membrane 212 may have elastic properties. For example, the occlusion membrane 212 may be capable of deformation with elongation at least between 100% and 1000% (e.g., 100% to 1000%, 200% to 800%, 400% to 600%, values between the foregoing, etc.). As described above, the electrospun membrane may comprise a polyurethane or polyurethane blend, which may have an ultimate strain at least between 350% and 600% (e.g., 350% and 600%, 400% and 500%, values between the foregoing, etc.). The electrospun fibers used for the electrospun membrane may include fibers with diameters at least between 750 nanometers micron and 5 microns (e.g., 750 nanometers micron to 5 microns, 2 microns to 4 microns, 2.5 microns to 3.5 microns, values between the foregoing, etc.). In this example, the occlusion membrane 212 may act as a frangible tissue scaffold. Depending on the type and diameter of the fibers used, the occlusion membrane 212 may preferably have a scaffold thickness between 50 microns and 200 microns (e.g., 50 microns and 200 microns, 75 microns and 175 microns, 100 microns and 150 microns, 100 microns and 125 microns, values between the foregoing, etc.). However, it is recognized that the thickness of the occlusion membrane 212 varies based on a number of factors and in some embodiments, it may be desirable to have an occlusion membrane 212 with a smaller or larger thickness. Use of a thin occlusion membrane 212 may provide a benefit of allowing the occlusion membrane 212 to lie flat against the septum such that the occlusion membrane 212 flexes with the septum once implanted. Generally, the fiber architecture of the occlusion membrane 212 is random non-aligned fibers, which gives the occlusion membrane 212 similar properties in all direction. The occlusion membrane 212 acting as a tissue scaffold may promote growth of a layer of tissue across at least the outer surface of the occlusion membrane 212 (e.g., in a direction facing the left atrial side 216). However, in some embodiments, the occlusion membrane 212 may be configured to limit or prevent any tissue ingrowth within the occlusion membrane 212 (e.g., the tissue scaffold) such that the occlusion membrane 212 maintains a similar elasticity with and without a tissue layer present. In some embodiments, the occlusion membrane 212 may be semi-porous. For example, the occlusion membrane 212 may allow gas to pass through it but may not allow liquid to pass through it. In some embodiments, the semi-porous membrane may allow water to pass through it but not blood. Use of a non-cell porous structure may allow for immediate septal occlusion once the occluding device 200 is implanted and prior to tissue growth. In some embodiments, as a result of the electrospinning process, the occlusion membrane 212 may be impermeable/non-porous to certain elements (e.g., endothelial cells) but may allow certain elements (e.g., fluids like plasma) to pass/diffuse across the membrane 212. As such, the electrospun occlusion membrane 212 creates an excellent scaffold for cell growth. Once the occluding device 200 is implanted, a medical instrument may cross directly through any tissue layer (e.g., tissue layer 115) on the occluding device 200 and the occlusion membrane 212, which may cause elastic deformation of the occlusion membrane 212. Generally, it may be desirable to avoid plastically deforming the occlusion membrane 212 during a procedure. When the subsequent medical procedure is completed and the medical instrument is retracted, the occlusion membrane 212 may return to roughly the original position (e.g., as a result of the occlusion membrane's 212 elastic properties) and continue to occlude the lumen 4. Generally, the occluding device 200 may provide a similar amount of occlusion of the lumen 4 after being crossed with a medical instrument as when the occluding device 200 is initially implanted (e.g., prior to being crossed). For example, the medical instrument may cause minimal damage or plastic deformation to the occlusion membrane 212. Additionally, after being crossed in a subsequent procedure (e.g., where a tissue layer is pierced), the occlusion membrane 212 may continue to promote new tissue growth across the occluding device 200, which will improve the occlusion of the occluding device 200 as the tissue layer grows.

FIGS. 2B1 and 2B2 illustrate left atrial side views of the occluding device 200 and FIG. 2C illustrates a back view of the occluding device 200. As shown in FIG. 2B1/2B2 and 2C, the front/left atrial side 216 of the occluding device 200 may present a large target for the operator (e.g., a physician) of a medical instrument to cross; however, the crossable portion/region of the occluding device 200 comprising the lumen 208 is smaller than the overall diameter of the left atrial side 216 of the occluding device 200, as shown in FIG. 2C.In some embodiments, the anchoring frame 201 supporting the membranes 210, 212 can be constructed from a radiopaque material. In some examples, the radiopaque material used can be nitinol. A radiopaque support structure 201 provides the physician with a clear visualization of the occluding device 200 within the anatomy when under fluoroscopic guidance. This feature is important both when implanting the occluding device 200, for accurate deployment and positioning, as well as for recrossing the occluding device 200 (acutely or in a chronic setting), as positioning of the septal puncture is much easier. In some embodiments, to assist the operator with crossing the occluding device 200, all or a portion of the occlusion membrane 212 may comprise a radiopaque material. For example, a portion of the occlusion membrane 212 may be dyed or treated in another manner is a radiopaque substance. For example, between 0% and 100% of the occlusion membrane 212 (e.g., 0% to 100%, 10% to 90%, 25% to 75%, 50% to 60%, values between the foregoing, etc.) may comprises a radiopaque material. In some embodiments, it may be preferable for less than 30% of the occlusion membrane 212 to comprise a radiopaque material. In some embodiments, the portion of the occlusion membrane 212 comprising a radiopaque material may correspond to the lumen 208. For example, a central portion (e.g., a circle) of the occlusion membrane 212 that is approximately the same size as the lumen 208 may comprise a radiopaque material. In some embodiments, the occlusion membrane 212 may only include a radiopaque target such as, for example, an x-shaped target, circle-shaped target, and/or the like. When a medical procedure involves crossing the occluding device 200, an operator of a medical instrument may use fluoroscopic imaging to identify the radiopaque portion of the occluding device 200 and the operator may use this location to safely cross the occluding device 200, without contacting the support structure 201. In some embodiments, the radiopaque portion of the occluding device 200 may allow an operator of a medical instrument to select a septal area to cross outside of the occluding device 200.

FIG. 2D illustrates a side view of the occluding device 200 and FIG. 2E illustrates a section view of the occluding device 200 along the line 2E-2E in FIG. 2B1. The left atrial side 216 of the occluding device 200, including the occlusion membrane 212, may comprise an approximately planar surface. In some embodiments, the outer edges 222 of the left atrial side 216 may curve in a direction away from the back/right atrial side 218. As shown more clearly in FIG. 2E, the outer edges' 222 inflection on the left atrial side 216 may only begin near the peripheral edges of the left atrial side 216. For example, between 80% and 100% of the diameter of the left atrial side 216 (e.g., 80% to 100%, 82.5% to 97.5%, 85% to 95%, 87.5% to 92.5%, values between the foregoing, etc.) may be approximately planar. Generally, it may be preferable for the outer edges 222 to be as flush with the septal tissue as possible without causing trauma to the tissue. For example, the outer edge 222 may be flat (i.e., no inflection) or may have a slight inward curve (i.e., away from the septal tissue) as shown in at least FIG. 2E. Additionally, flat or minimally curved outer edges 222 may ensure apposition with the septal tissue and subsequently ensure good anchorage and rapid endothelization as a result. In some embodiments, an occluding device 200 with curved outer edges 222 may improve the delivery of the occluding device 200, as the occluding device 200 may be less likely to scratch or contact the internal lumen of the delivery sheath. The right atrial side 218 of the occluding device 200 may also be approximately planar and may be approximately parallel to the left atrial side 216. For example, a medical instrument in the right atrium 101 may pass through the right atrial side 218, crossing through a first plane (e.g., corresponding to the right atrial side 218) and a second parallel plane (e.g., corresponding to the left atrial side 216) into the left atrium 102. In some embodiments, the second anchoring portion 204 may be configured to be flat against the septal tissue once implanted, which may result in a flattened radius of curvature in the support structure 201. In some examples, the occluding device 200 may be configured such that minimal or no discontinuities exist within the two planes defined by the left atrial side 216 and the right atrial side 218. For example, one or both the left atrial side 216 and the 218 may form a continuous surface. For example, portions of the support structure 201 may extend minimally into the left atrium 102 from the left atrial side 216 and minimally into the right atrium 101 from the right atrial side 218. For example, minimal extension may be between 0 mm and 0.5 mm (e.g., between 0 mm and 0.5 mm, 0.1 mm and 0.4 mm, 0.2 mm and 0.3 mm, values between the foregoing, etc.). For further clarity, discontinuities may refer to portions of the occluding device 200 that are out of plane of the majority of the left atrial side 216 or out of plane of a majority of the right atrial side 218, once the occluding device 200 implanted. For example, with respect to the left atrial side 216, the outer edge 220 and the curved portion of the outer edges 222 may extend less than 0.5 mm, and preferably less than 0.1 mm into the left atrium 102 with respect to the plane defined by left atrium 102 wall. With respected to the right atrial side 218, the outer petal tips 244 may extend less than 0.5 mm, and preferably less than 0.1 mm into the right atrium 101 with respect to the plane defined by the right atrium 101 wall. The minimal extension of the occluding device 200 may provide certain benefits, such as allowing the left atrial side 216 and the right atrial side 218 to have a flat profile against the septum walls. For example, a flat profile may improve the speed at which the occluding device 200 is covered with endothelium because the tissue does not need to grow over edges or only needs to grow over small edges of the occluding device 200. An additional benefit of eliminating/reducing the discontinuities of the occluding device 200 is that discontinuities are often sites for thrombus formation and infection.

With reference to FIG. 2D, the occluding device 200 may be approximately bowl shaped when viewed from the side. As shown in FIG. 2E, the first and second anchoring portions 202, 204 delimit the lumen 208, with the first anchoring portion 202 curving away from the central portion 206 in a direction towards the left atrial side 216 and away from the lumen 208 and the second anchoring portion 204 curving away from the central portion 206 in a direction towards the right atrial side 218 and away from the lumen 208. As the first anchoring portion 202 continues away from the lumen 208, the first anchoring portion 202 forms the first plane 216 described above. As the second anchoring portion 204 continues away from lumen 208, a bottom edge of the second anchoring portion 204 contacts the second plane 218. Continuing away from the lumen 208, the second anchoring portion 204 extends in a direction towards the left atrial side 216 at an angle between 5% and 85% (e.g., 5% to 85,15% to 75%, 25% to 60%, 35% to 55%, values between the foregoing, etc.) relative to the second plane corresponding to the right atrial side 218. The second anchoring portion 204 may extend in this direction until reaching an outer edge 220 of the second anchoring portion 204. In some embodiments, the outer edge 220 may have a different angle relative to the second plane of the right atrial side 218 than the majority of the second anchoring portion 204. The frame covering membrane 210 conforms to the shape of the support structure 201 in the expanded configuration and may extend from a position at or near the outer edge 220 of the second anchoring portion 204, along the curve of the second anchoring portion 204, across the central portion 206 and to a position at or near the outer edges 222 of the first anchoring portion 202. The bowl shape of the occluding device 200 may reduce the presence of protrusions into the left atrium 102 once the occluding device 200 is implanted, which may be beneficial because protrusions into the left atrium 102 may increase the risk of stroke. In some embodiments, it may be preferable to reduce the corresponding bulge in the right atrium 101 caused by the bowl shaped occluding device 200.

In some embodiments, the occluding device 200 may include a hole in the occlusion membrane 212 such that the occluding device 200 behaves as a compliant shunt. For example, the hole may be located near the center of the occlusion membrane 212. Due in part to the compliant nature of the occlusion membrane 212, the hole may enlarge with increasing pressure differentials across the septum, resulting in the occluding device 200 behaving like a dynamic pressure release valve. One benefit of this embodiment may be improved performance of heart failure patients during exercise.

b. Membrane Properties

As described above, the occluding device 200 may include one or more membranes, such as the frame covering membrane 210 and the occlusion membrane 212. While some properties of the membranes 210, 212 are described above, for greater clarity, additional membrane behavior and properties are described in this section. It is recognized that the properties described herein can apply to either one or both the frame covering membrane 210 and the occlusion membrane 212. Within this section, the properties of the membranes 210, 212 will be described. It is further recognized that while some embodiments of the occluding device 200 may include all the following and aforementioned properties, other embodiments of the occluding device 200 may include only some of the properties described herein.

The membranes 210, 212 may comprise an electrospun membrane such as, a continuous micro/nano polymer fiber. The electrospun fibers used for the electrospun membranes 110, 112 may include fibers with diameters at least between 0.5 microns and 5 microns (e.g., 0.5 microns to 5 microns, 1 microns to 4.5 microns, 1.5 microns to 4 microns, 2 microns to 3 microns, values between the foregoing, etc.). In some cases, it may be preferable for the fibers of the electrospun membranes 110, 112 to be between 1-3 microns in diameter. As noted above, use of an electrospun membrane may result in the membranes 210, 212 having high elasticity and/or being highly compliant. Additionally, the electrospun membranes 110, 112 may allow for a thin layer of tissue to endothelialize on the occluding device 200 once implanted. Further, the electrospun membranes 110, 112 may improve the septal compliance of the occluding device 200. A compliant septum better mimics a natural septum, which allows blood pressure within the heart to better mimic natural physiology.

The compliant nature of the occluding device 200 may be attributed in part to the elastic properties of the electrospun membranes 110, 112. For example, the electrospun membranes 110, 112 may be capable of deformation with elongation at least between 100% and 1000% (e.g., 100% to 1000%, 200% to 800%, 400% to 600%, values between the foregoing, etc.). In some examples, it may be preferable for the electrospun membranes 110, 112 to be configured to stretch more than 400% during a routine procedure (e.g., being punctured and crossed with a medical tool, such as a catheter) without plastically deforming. This range of elongation may allow the occluding device 200 to sufficiently occlude the defect 107 while the occluding device 200 is being punctured (e.g., because the fibers are stretching and surrounding the medical tool) as well as immediately after the medical tool is removed (e.g., because the fibers were elastically stretched and can return to their original configuration). The ultimate strain of the fibers of the electrospun membranes 110, 112 may be least between 350% to 600% (e.g., 350% to 600%, 400% to 550%, 450% to 500%, values between the foregoing, etc.).

Because the electrospun membranes 110, 112 are elastic, the electrospun membranes 110, 112 can flex during the cardiac cycle. During a normal cardiac cycle, the septum flexes with the changing pressure in the heart (e.g., in a direction towards the left atrium and in an opposite direction towards the right atrium) continuously. Once implanted, it is desirable for the occluding device 200 to not hinder or significantly hinder the normal flexion of the septum. Rather, it is desirable to promote normal flexion in the septum. In some cases, once the occluding device 200 is implanted, the occluding device 200 may move with the septum during a normal cardiac cycle. Further, the electrospun membranes 110, 112 may deflect further into the left atrium and into the right atrium, depending on the stage of the cardiac cycle. In some examples, the electrospun membranes 110, 112 can elastically deflect (e.g., relative to the support structure 201) at least between 0.5 mm to 5 mm (e.g., 0.5 mm to 5 mm, 1 mm to 4.5 mm, 1.5 mm to 4 mm, 2 mm to 3.5 mm, 2.5 mm to 3 mm, values between the foregoing, etc.). It is recognized the amount of deflection will vary between patients and the amount of deflection is dependent in part on the pressure in the patient's heart. In this manner, the normal flexion of the septum is promoted, and the strain placed on the septum by the occluding device 200 is minimized. This behavior can result in significant long term benefits when compared to other septal occluding devices that are rigid. Rigid occluding devices place strain on the septum and the heart and may result in a stiff septum that does not flex normally during the cardiac cycle, which may impact a patient's hemodynamics and can result in long term complications (e.g., increased risk of stroke, long term care requirement, erosions of adjacent cardiac structures, atrial arrythmias, etc.).

The ability of the occluding device 200 to flex with the septum and promote normal cardiac behavior may be attributed in part to weight of the occluding device 200. The lightweight nature of the occluding device 200 may preserve septal compliance and protect the surrounding structures of the septum and the heart. For example, a lighter occluding device 200 may place less stain on the septum once implanted. In another example, a lightweight occluding device 200 may move freely with the septums during flexion. In some cases, the occluding device 200 may weigh between 25 micrograms to 200 micrograms (e.g., 25 μg to 200 μg, 50 μg to 150 μg, 75 μg to 125, values between the foregoing, etc.). In some cases, it may be preferable for the occluding device 200 to weigh less than 50 micrograms. It is recognized that the weight of the occluding device 200 is dependent in part on the size of the occluding device 200 and the size of the defect 107 that the occluding device 200 is being used to occlude. For example, an occluding device 200 used for a small defect in a child patient may weigh less than an occluding device 200 used for a large defect in an adult patient. The above ranges may be for an occluding device 200 for an adult. In some cases, the occluding device 200 may be between 5-10 times lighter than commercial occluding devices currently on the market. The lightweight nature of the occluding device 200 may be attributed in part to the use of the electrospun fibers for the membranes 110, 112. In some commercial occluders, a metal braid or structure is used to occlude defects in the septum. Use of a metal braid may increase the weight and/or stiffness of the occlusion device as well as result in other negative consequences described herein. For example, heavy/stiff occlusions devices may cause disruptions to the septal conduction system. The lightweight nature of the occluding device 200 may also be attributed in part to the design of the first anchoring portion 202 and the second anchoring portion 204, which can be grasped on the outside of the support structure 201 during implantation. The support structure 201 is described further herein with reference to at least FIG. 3A-3D.

The compliant nature of the occluding device 200 may be attributed in part to the thickness T of the occluding device 200 (see e.g., FIG. 2D). For example, it may be desirable to minimize the thickness T of the occluding device 200 to reduce the extension of the occluding device 200 into the left and right atriums 102, 101. In the heart, the septum may have a thickness of approximately 1 mm-5 mm (depending on the patient and the position of the defect) where the occluding device 200 is to be implanted. In some examples, the occluding device 200 may have a thickness T of between, 1 mm to 5 mm, 2 mm to 5 mm (e.g., 1 mm to 5 mm, 2 mm to 5 mm, 2.5 mm to 4.5 mm, 3 mm to 4 mm, values between the foregoing, etc.). In some cases, it may be desirable for the occluding device 200 to be 5 mm thick or less. The occluding device 200 may be substantially thinner than commercial occluding devices, which can have a thickness of 5 mm-10 mm or greater.

As shown in FIG. 4B, the implanted occluding device 200 (pre endothelization) has a similar thickness to the partition 7. Additionally, the occluding device 200 extends minimally into the right atrium 101 and the left atrium 102 once implanted with no discontinuities which can be sites for thrombus formation and infection. Specifically, the occluding device 200 is substantially flush with the left atrium 102. Substantially flush, as used here, may mean that the occluding device 200 extends no more than 0.5 mm into the left atrium 102 relative to the edge of the partition 7. For example, in some cases, the occluding device 200 may extend into the left atrium 102 between 0.05 mm to 0.5 mm (e.g., 0.05 mm to 0.5 mm, 0.1 mm, to 0.4 mm, 0.15 mm to 0.35 mm, 0.2 mm to 0.3 mm, values between the foregoing, etc.). In some cases, it may be desirable to extend less than 0.1 mm into the left atrium 102. Minimal extension of the occluding device 200 into the left atrium 102 may reduce/remove the long-term medical therapy and associated morbidity traditionally associated with septal occluding devices in part because the occluding device 200 places a substantially lower strain on the septum. Additionally, once there is endothelization of the occluding device 200 (see e.g., FIG. 4C), the occluding device 200 may be sufficiently embedded in the septum and covered in tissue that the septum profile in the left atrium 102 is substantially flat. Similarly, the occluding device 200 may also extend minimally into the right atrium 101, even before being covered in tissue after endothelialization. For example, in some cases, the occluding device 200 may extend into the right atrium 101 (relative to the partition 7) between 0.1 mm to 2 mm (e.g., 0.1 mm to 2 mm, 0.25 mm, to 1.75 mm, 0.5 mm to 1.5 mm, 0.75 mm to 1.25 mm, values between the foregoing, etc.). Comparatively, commercial occluding devices may extend between 3 and 5 mm (or greater) into the each of the right and left atriums 101, 102. While a small overall thickness and minimal extension into the right and left atriums 101, 102 may be desirable for improved septal compliance and improved long-term patient health, an additional benefit is the rapid endothelialization of the occluding device 200. For example, the occluding device 200 may substantially endothelialize faster than commercial occluding devices (which may take years to even begin to endothelialize) due at least in part to the ultra-thin profile and minimal atrial extension. A smooth tissue interface results in a corresponding reduction in stroke risk because a smooth endothelium reduces the shear forces on the septum which helps to prevent thrombus formation.

For further clarity, current commercial devices present a number of drawbacks in comparison with the occluding device 200 in terms rate of endothelialization and consequences of slow endothelialization. For example, when a patient has a currently available commercial occluding device implanted, the patient is often required to take at least 3-6 months of anti-platelet and antibiotic medication while the device and implantation site heals and the device begins to be covered with endothelium. At least during this time period, and sometimes for greater time periods, the current devices have exposed metal (e.g., braids) in the blood stream, which can be sites for thrombus formation and/or infection. The extended period before current device are covered with endothelium can be attributed in part at least three factors. First, current devices typically use metal (e.g., metal braids), and often unencapsulated metal, which typically endothelializes at a slower rate in comparison to compliant mesh. Second, current devices have a thick profile, and therefore require tissue growth up and over the sides of the device. Third, current devices have a number of discontinues (i.e., portions of the device that extend/protrude directly into the blood stream. Because of the discontinuities, the protrusions take a long time to cover with tissue and in many cases, are never covered with tissue. An additional suggested reason for the improved endothelialization of the occluding device 200 concerns the relative motion between adjacent parts of the device. In the case of commercial braided devices, the filaments continuously move relative to one another, which may increase the time for cell growth to cover and may increase the inflammation and scarring response. Conversely, there is limited relative motion between adjacent parts of the occluding device 200, which may contribute to the improved endothelialization.

In another example, the compliant nature of the occluding device 200 may be attributed in part to the thickness T2 of the lumen 208 of occluding device 200 (see e.g., FIG. 2E). In this example, the thickness T2 may encompass the thickness of the occlusion membrane 212 as well as any tissue that ultimately forms in the lumen 208 after the occluding device 200 is implanted. While the lumen may be open when the occluding device 200 is first implanted, overtime, tissue may form and fill the lumen 208. Depending on the type and diameter of the fibers used, the occlusion membrane 212 may preferably have a scaffold thickness between 20 and 100 microns (e.g., 20 microns and 100 microns, 30 microns and 90 microns, 40 microns and 80 microns, 50 microns and 70 microns, 50 microns and 60 microns, values between the foregoing, etc.). It may be desirable to minimize the thickness T2 of the lumen 208 to reduce the extension of the occluding device 200 into the left and right atriums 102, 101. As noted above, the septum may have a thickness of approximately 2 mm-2.5 mm (depending on the patient) where the occluding device 200 is to be implanted. In some examples, the lumen 208 may have a thickness T2 of between, 1 mm to 5 mm, 2 mm to 5 mm (e.g., 1 mm to 5 mm, 2 mm to 5 mm, 2.5 mm to 4.5 mm, 3 mm to 4 mm, values between the foregoing, etc.). In some cases, it may be desirable for the thickness T2 of the lumen 208 to be 3 mm or less.

As described herein, the occluding device 200 and specifically the membranes 210, 212 can be configured to promote rapid tissue growth across the membranes 210, 212 and/or around the occluding device 200. Once implanted, the membranes 210, 212 may act as scaffold for cell growth. For example, a full tissue layer (e.g., covering at least a majority of the occlusion membrane 212) may form after a certain period of day, such as, for example, between 10 days and 90 days (e.g., 10 days and 90 days, 20 days and 80 days, 30 days and 70 days, 40 days and 60 days, 45 days and 55 days, values between the foregoing, etc.) after implantation of the occluding device 200. Is some embodiments, there can be 90% tissue coverage over the portions of the membranes 210, 212 on first anchoring portion 202 (i.e., the portion extending into the left atrium 102) within 10 days. In some embodiments, there can be 80% tissue coverage over the portions of the membranes 210, 212 on the first anchoring portion 202 within 10 days. It is recognized that the period of time to support a full tissue layer may vary based on a number of factor including, the patient, the size of the defect, the size of the occluding device 200, and/or the like. The rapid tissue growth may be as result of the one or more of the membrane properties/features described above. In particular, the rapid growth may be a result of the use of an electrospun membrane which can result in the formation of a complete endothelial monolayer significantly faster when compared to ePTFE membranes. In some examples, the rapid tissue growth can be a result the membranes 210, 212 comprising one or more of the following features: a continuous micro/nano polymer fiber, fiber diameters in a range of 0.5 microns to 5 microns, fibers with elongation at least between 100% and 1000%, fibers with an ultimate strain between 350% and 600%. In certain embodiments, the rapid tissue growth can be a result of the membranes 210, 212 having one or more of the following ranges in combination: fiber diameters between 0.5 microns to 5 microns, fiber diameters between 1 and 3 microns, fiber capable of deformation with elongation between 100% and 500%, fiber capable of deformation with elongation between 200% and 400%, fibers with an ultimate strain between 350% and 600%, fibers with an ultimate strain between 400% and 500%. Note that these ranges can include additional ranges as described above and can be used with combinations of other features in the application (e.g., the support structure 201 features, the delivery system 500 features, the other occluding device 200 features, and/or the other membrane 210, 212 features described herein). As a result of the rapid tissue formation, the interatrial blood flow between the left atrium 102 a right atrium 101 through the lumen 4 may have been completely eliminated or further reduced relative to the intra-operative state (e.g., as described with reference to FIG. 4B).

c. Occluding Device Frame

FIGS. 3A-3D illustrate the frame or support structure 201 of the occluding device 200 in an expanded configuration. FIG. 3A illustrates a perspective view of the support structure 201 and FIG. 3B illustrates a left atrial side view of support structure 201. As described above, the support structure 201 comprises a first anchoring portion 202 and a second anchoring portion 204 which extend radially outwards from a central portion 206. In some embodiments, the first anchoring portion 202 and the second anchoring portion 204 may comprise separate components joined (e.g., welded, hinged) at the central portions 206, while in other embodiments, the first and second anchoring portions 202, 204 comprises one component, shaped to form the support structure 201. The central portion 206 may comprise a plurality of central connection portions (e.g., 206A, 206B, 206C, etc.), which may define a boundary of the lumen 208.

The first anchoring portion 202 may comprise a plurality of interconnected petals 228, wherein individual petals 228 are formed from struts 224. For example, a first petal 228A comprises a first strut 224A and a second strut 224B. In some embodiments, the first anchoring portion 202 may comprise 2, 4, 6, 8, 10, 12, 14, 16, 18, 20, and/or the like petals 228. Individual struts 224 may extend radially outwards from the central portion 206 in a direction towards the left atrial side 216 before forming the plane corresponding to the left atrial side 216. Two struts 224 forming a petal 228 may be joined at a petal tip 230. The petal tip 230 may comprise a half loop and may extend in a direction away from the lumen 208 and the right atrial side 218 at an angle relative to the left atrial side 216 as described above. For example, the first strut 224A and the second strut 224B are connected at one end at a first central connector 206A (e.g., forming the central portion 206) and are connected at an opposite end at a first petal tip 230A. As two struts 224 forming a petal 228 extend away from the central portion 206, the distance between the two struts 224 increases until a maximum distance is reached near the center of the petal 228. After reaching the maximum distance, the distance between two struts 224 decreases until reaching the petal tip 230. Individual petals 228 may be connected to adjoining petals 228 on each side by petal connectors 226. Individual petals 228 may be connected to adjoining petals 228 at or near the location where the two struts 224 forming the individual petal 228 are at a maximum distance from each other. For example, the first petal 228A comprising first strut 224A and second strut 224B is connected at a first petal connector 226A to a second petal 228B comprising a third strut 224C and a fourth strut 224D. Similarly, the first petal 228A is connected at a second petal connector 226B to a third petal 228C comprising a fifth strut 224E and a sixth strut 224F. FIG. 3B illustrates a top view of the support structure 201 of the occluding device 200, further illustrating the components discussed above.

FIG. 3C illustrates a right atrial side view of the support structure 201 of the occluding device 200 and FIG. 3D illustrates a side view of the support structure 201. The second anchoring portion 204 may comprise a plurality of interconnected inner petals 232 and outer petals 234, where individual inner petals 232 are formed from struts 236 and individual outer petals 234 are formed from struts 236. Further, adjacent struts 236 of two adjacent inner petals 232 form the outer petals 234. For example, a first inner petal 232A comprises a first strut 236A and a second strut 236B and a second inner petal 232B comprises a third strut 236C and a fourth strut 236D, while a first outer petal 234A comprises the second strut 236B and the third strut 236C. Generally, the second anchoring portion 204 comprises the same number of inner petals 232 as outer petals 234. In some embodiments, the second anchoring portion 204 may comprise 2, 4, 6, 8, 10, 15, 20, 25, 30, and/or the like inner petals 232 and outer petals 234.

Individual struts 236 forming inner petals 232 may extend radially outwards from the central portion 206 in a direction towards the right atrial side 218 before curving upwards towards the left atrial side 216. The base of the curve of individual struts 236 forming an inner petal 232 may contact the plane corresponding to the right atrial side 218. Two struts 236 forming an inner petal 232 may be joined at an inner petal tip 238. A plurality of petal tips 238 may form outer edge 220 of the second anchoring portion 204 which may correspond to the outer edge of a frame covering membrane 210. The inner petal tips 238 may comprise the portions of the second anchoring portion 204 that are closest to the left atrial side 216 when the occluding device 200 is in an expanded configuration. For example, the first strut 236A and the second strut 236B are connected at one end at the first central connector 206A and are connected at an opposite end at a first inner petal tip 238A. As two struts 236 extend away from the central portion 206, the distance between the two struts 236 increases until a maximum distance is reached near the center of the inner petal 232. After reaching the maximum distance, the distance between two struts 236 decreases until reaching the inner petal tip 238. Individual inner petals 232 may be connected to adjoining inner petals 232 on each side by inner petal connectors 240. Individual inner petals 232 may be connected to adjoining inner petals 232 at or near the location where the two struts 236 forming the individual inner petal 232 are at a maximum distance from each other. For example, the first inner petal 232A comprising the first struts 236A and the second strut 236B is connected at a first inner petal connector 240A to the second inner petal 232B comprising the third strut 236C and the fourth strut 236D. Similarly, the first inner petal 232A is connected at a second inner petal connector 240B to a third inner petal 232C comprising a fifth strut 236E and a sixth strut 236F.

Individual struts 236 forming outer petals 234 may extends radially outwards from the central portion 206 in a direction towards the right atrial side 218 before reaching an inflection point 242. At the inflection point 242, the individual struts 236 forming outer petals 234 may curve back towards the central portion 206, continuing in a direction towards the right atrial side 218. The curvature of the outer petals 234 is shown most clearly in FIG. 3D. Two struts 236 forming an outer petal 234 may be joined at an outer petal tip 244. The outer petal tip 244 may comprise a loop structure with an open interior. In some embodiments, portions of the two struts 236 forming an outer petal 242 may form the outer petal tip 244. As described further herein, the outer petal tips 244 may be used to connect the occluding device 200 to a medical instrument for implanting the occluding device 200. For example, the outer petal tips 244 may include or define eyelets for connecting the occluding device 200 to a delivery system. In some embodiments, the outer petal tip 244 may terminate at or near the second plane corresponding to the right atrial side 218. As described above, outer petals 234 may comprise the struts 236 of adjacent inner petals 232. For example, the first outer petal 234A comprises the second strut 236B and the third strut 236C, and a second outer petal 234B comprises the first struts 236A and the sixth strut 236F. As two struts 236 (e.g., struts 236B and 236C) forming an outer petal 234 (e.g., outer petal 234A) extend away from the inner petal connector (e.g., the first inner petal connector 240A), the distance between the two struts 236 increases until a maximum distance is reached at or near the inner petal tips 238 corresponding to the two inner petals 232 (e.g., first inner petal tip 238A and second inner petal tip 238B). After reaching the maximum distance, the distance between two struts 236 decreases until reaching the outer petal tip 244 (e.g., outer petal tip 244A).

In some embodiments, the first anchoring portion 202 and the second anchoring portion 204 may be aligned such that the petal tips 230 of the first anchoring portion 202 are offset from the outer petal tips 244 of the second anchoring portion 204. For example, the petal tips 230 of the first anchoring portion 202 may be approximately axially aligned relative to the lumen 208 with the inner petal tips 238 of the second anchoring portion 204. Similarly, the outer petal tips 244 of the second anchoring portion 204 may be approximately axially aligned relative to the lumen 208 with the petal connectors 226 of the first anchoring portion 202. This configuration may improve the compressive force applied to the partition 7 when the occluding device 200 is implanted.

As shown in FIG. 3B, the support structure 201 in the expanded configuration includes an inner diameter D1 and an outer diameter D2. The inner diameter D1 may be defined by the distance between two opposing central connectors 206 (e.g., central connector 206A and central connector 206D). The size of the inner diameter D1 may be slightly larger or slightly smaller than the lumen 4 corresponding to a defect 107. The outer diameter D2 may be defined by the distance between two opposing petal tips 230 of the first anchoring portion 202 (e.g., petal tip 230A and petal tip 230D. The number of petals 228 of the first anchoring portion 202, the number of inner petals 232 and outer petals 234 of the second anchoring portion 204, the length of struts 224 and struts 236, and the inner and outer diameters D1, D2 of the support structure 201 can be varied as need for particular application of the occluding device 200.

In some embodiments, the inner diameter D1 can be between 4 mm and 45 mm (e.g., 4 mm to 45 mm, 10 mm to 40 mm, 20 mm to 30 mm, values between the foregoing, etc.). The inner diameter D1 of a particular occluding device 200 may be selected based on the size of a target defect 107. In some embodiments, the outer diameter D2 can be between 10 mm and 60 mm (e.g., 10 mm to 60 mm, 15 mm to 50 mm, 20 mm to 45 mm, 25 mm to 35 mm, values between the foregoing, etc.). Generally, the outer diameter D2 size may vary based on a desired inner diameter D1 size, and the inner and outer diameters D1, D2 may be selected based on the size of a target defect 107. The outer diameter D2 is generally larger than the target defect 107 such that there is enough tissue in the partition 7 for the support structure 201 to compress therebetween.

As described herein, the support structure 201 is configured to expand and contract between a compressed tubular configuration for insertion through a patient's vasculature and an expanded or extended configuration in which the first and second anchoring portions 202, 204, extend radially outwards from the central portion 206. The support structure 201 may comprise any suitable material that allows it to conform to a compressed tubular configuration with minimal plastic deformation and expand to an expanded configuration for implantation. The material may allow the support structure 201 to be self-expandable. Generally, the support structure 201 may comprise a non-toxic material to allow the occluding device 200 to be implanted in a human (e.g., a heart) without negative effects resulting from corrosion on the support structure 201. Further, the support structure 201 may be configured to protect the surrounding tissue once implanted. In some embodiments, the support structure 201 may comprise a metal, a plastic, bioresorbable material, and/or the like. In some embodiments, the support structure 201 may comprise a metal, a plastic, and/or the like. In some embodiments, the support structure 201 may comprise a shape-memory material, such as, for example, Nitinol. A shape-memory material may allow the support structure 201 to conform to the tubular configuration and self-expand once released from the implantation device. In some embodiments, the support structure 201 may comprise a lightweight polymer covered Nitinol.

### III. Occluding Device Implantation

FIGS. 4A-4C illustrate a cross-section of a partition of the heart prior to implanting an occluding device 200, during implantation of the occluding device 200, and post-implantation of the occluding device 200. FIG. 4A illustrates a cross-sectional view of the partition 7 of the heart 103 in a pre-operative state (i.e., prior to implantation of the occluding device 200). The partition 7 may correspond to the atrial partition 100 and may separate the right atrium 101 from the left atrium 102. As shown, the partition 7 includes a defect 107 with a lumen 4 extending through the partition 7. Prior to implanting the occluding device 200, blood may freely flow through the lumen 4 from the right atrium to the left atrium and vice-versa.

FIG. 4B illustrates a cross-section view of the partition 7 of the heart 103 in an intra-operative state, where the occluding device 200 has been implanted. For example, the occluding device 200 may be anchored in the fossa ovalis. During an operation, as described further herein, the occluding device 200 was positioned within the lumen 4 in a compressed configuration and expanded to an expanded configuration such that the first anchoring portion 202 is positioned on the left atrium 102 side of the partition 7 and the second anchoring portion 204 is positioned on the heart 103 of the partition 7. In the implanted state, the tissue of the partition 7 is compressed between the first anchoring portion 202 and the second anchoring portion 204, and the lumen 4 is aligned with the lumen 208 of the occluding device 200. Compressing the partition 7 allows the occluding device 200 to be fixed in its implanted state. In this configuration, the occlusion membrane 212 closes a present defect, occludes the lumen 4, and greatly reduces or eliminates the interatrial blood flow.

FIG. 4C illustrates a cross-section view of the partition 7 of the heart 103 in a post-operative state, where the occluding device 200 was implanted in the heart 103 in a prior operation. As shown, a layer of tissue 115 has grown over the occluding device 200, particularly on the left atrial side 216 on the occlusion membrane 212. In FIG. 4C, there is complete endothelization of the cell scaffold corresponding to the occluding device 200. As a result of the tissue layer 115, the interatrial blood flow between the left atrium 102 a right atrium 101 through the lumen 4 may have been completely eliminated or further reduced relative to the intra-operative state described in FIG. 4B. As described above, use of an electrospun material for the occlusion membrane 212 and/or the frame covering membrane 210 may have promoted the growth of the tissue layer 115. In some embodiments, a full tissue layer (e.g., covering the majority of the occlusion membrane 212) may form after a certain period of day, such as, for example, 1 day, 3 days, 5 day, 10 days, 15 days, 30 days, 45 days, 60 days, 90 days, and/or the like after implantation of the occluding device 200. It is recognized that the period of time to support a full tissue layer may vary based on a number of factor including, the patient, the size of the defect, the size of the occluding device 200, and/or the like. Use of an electrospun membrane may result in the formation of a complete endothelial monolayer significantly faster when compared to ePTFE membranes. For example, an ePTFE membrane may remain largely uncovered by cell tissue, even months after implantation. In the post-operative state with a full tissue layer 115, in order to cross the occluding device 200 with a medical instrument (e.g., from the left atrium 102 to the right atrium 101), an operator may pierce the tissue layer 115 and the occlusion membrane 212 to pass through the lumen 208. Once the subsequent procedure is completed and the medical instrument is retracted through lumen 208, the occlusion membrane 212 may continue to partially or completely occlude the lumen 4. Following the second operation, the tissue layer 115 will reform over the occluding device 200 until the lumen 4 is completely occluded again.

Any of the devices described herein (e.g., the occluding device 200, the occluding device 200′, the occluding device 300, devices including the frame 201″ or the frame 301′, and/or the like) can include one or more membranes that are identical or include some or all of the properties as the frame covering membrane 210 and/or the occlusion membrane 212 described herein. Additionally, the membranes described herein (e.g., the frame covering membrane 210, the occlusion membrane 212, etc.) can be used with other medical devices and implants.

### IV. Delivery System

a. Delivery System Overview

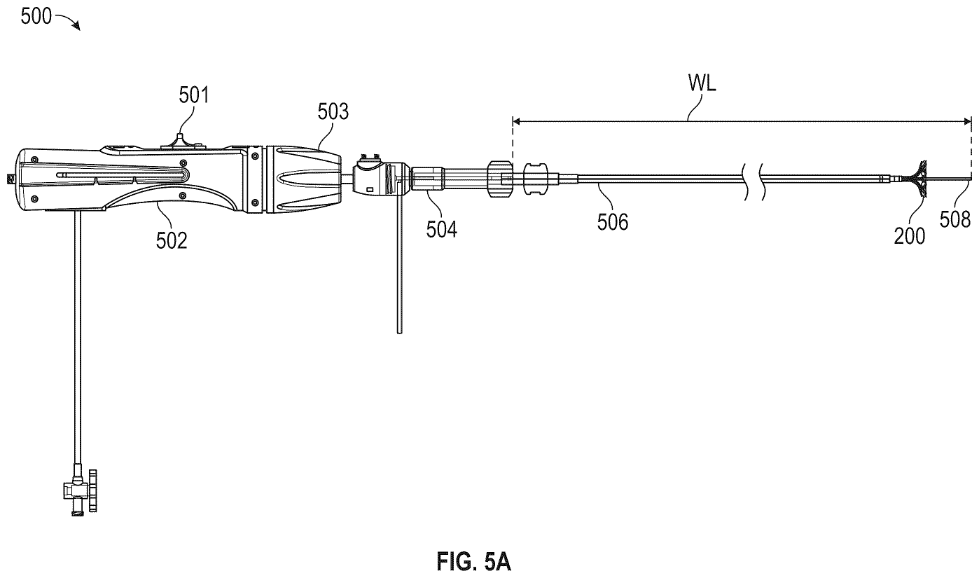

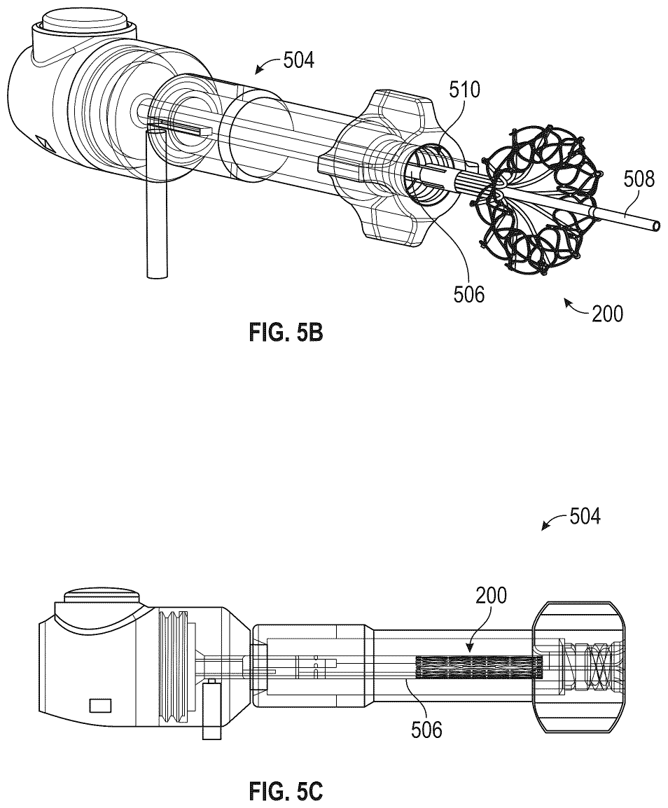

FIG. 5A illustrates an embodiment of a delivery system 500 that may be used to implant an occluding device 200 in a patient's heart. For example, the delivery system 500 may comprise a catheter system. The delivery system 500 may be an over-the-wire, single use, transfemoral delivery system that facilitates the attachment, loading, delivery, and deployment of the occluding device 200 for treatment of, for example, ostium secundum ASD. The delivery system 500 may include a handle 502, an implant loader 504, a Sheath 506 (e.g., 12 FR), and a catheter 508. In some embodiments, the delivery system 500 may be used with one or more of: a dilator, a guidewire (e.g., ultra-stiff guidewire), heparinized saline, a luer lock flushing syringe, and/or the like ancillary devices not shown. The handle 502 may include one or more mechanical systems for controlling the positioning, deployment, and release of the occluding device 200. For example, in the embodiment illustrated in FIG. 5A, the handle 502 includes a release button 501 and a rotation knob 503. The delivery system 500 may also include a grasping system 550 described further below. The release button 501 may be configured to move distally and proximally along the handle 502 to control the detachment of the occluding device 200 from the grasping system 550. For example, when the release button 501 is in a proximal position, the grasping system 550 may be locked and movement of the release button 501 to the distal position may unlock the grasping system 550 and permit detachment of the occluding device 200. The rotation knob 503 may be configured to rotate about an axis of the handle 502. The rotation knob 503 may control distal and proximal movement of the sheath 506. For example, rotation the rotation knob 503 in a first direction (e.g., clockwise) may cause the sheath 506 to retract proximally and rotation of the rotation knob 503 in a second direction (e.g., counterclockwise) may cause the sheath 506 to extend distally. As described further herein, the sheath 506 may be retracted prior to deployment of the occluding device 200.

As shown in FIG. 5A, an embodiment of the occluding device 200 is positioned near the end of the sheath 506 in an expanded configuration. In an operation, the occluding device 200 may be contained within the sheath 506 in a compressed configuration for delivery through the patient's vasculature towards the heart. In some embodiments the working length WL of the delivery system 500 may extend from the proximal end of the sheath 506 to the distal end of the catheter 508. The working length WL may be between 50 cm and 200 cm (e.g., 50 cm to 200 cm, 90 cm to 180 cm, 120 cm to 160 cm, values between the foregoing, etc.). It is recognized that the delivery system 500 is one example of a delivery system and any suitable delivery system can be used to implant the occluding device 200.

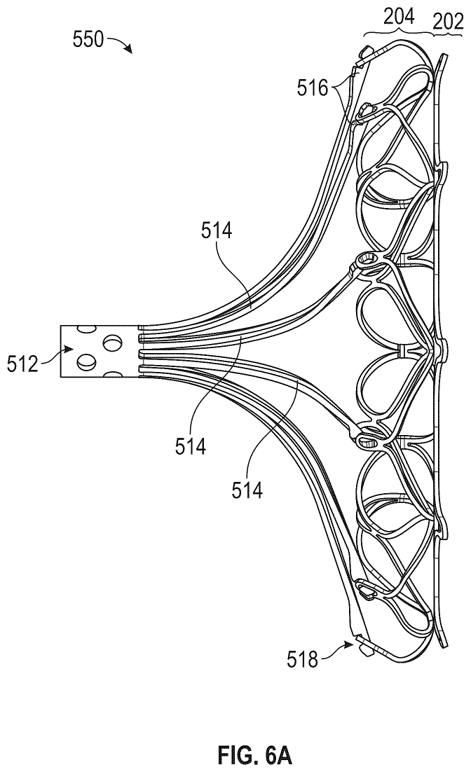

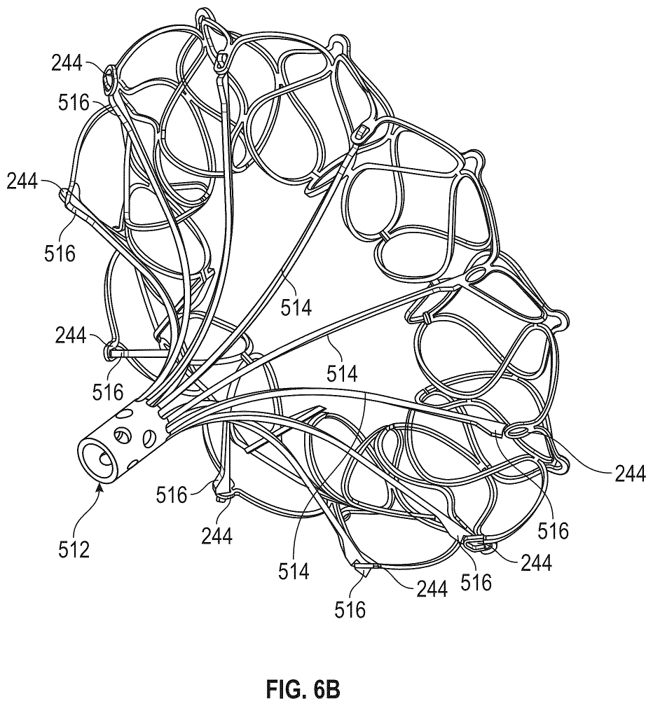

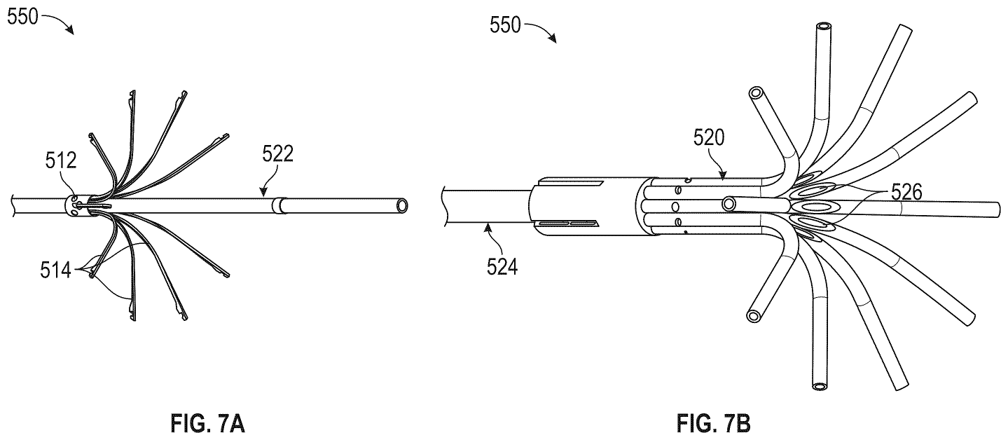

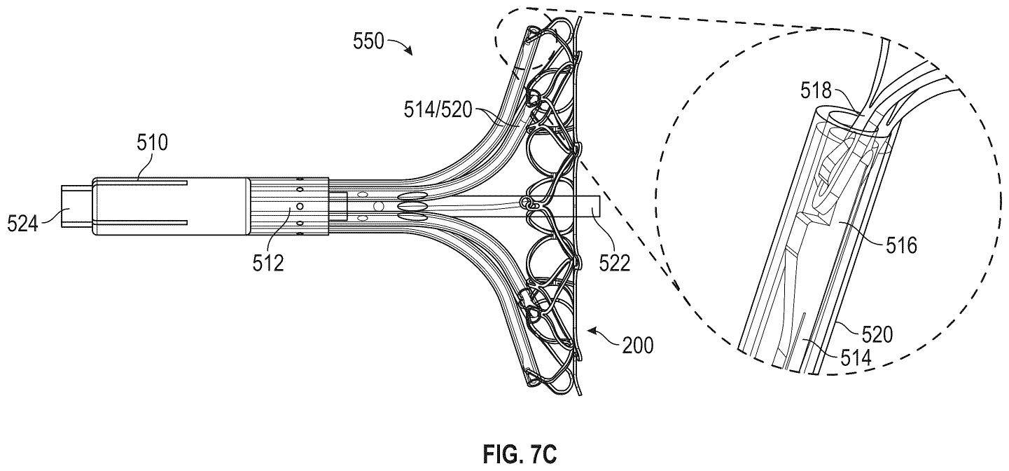

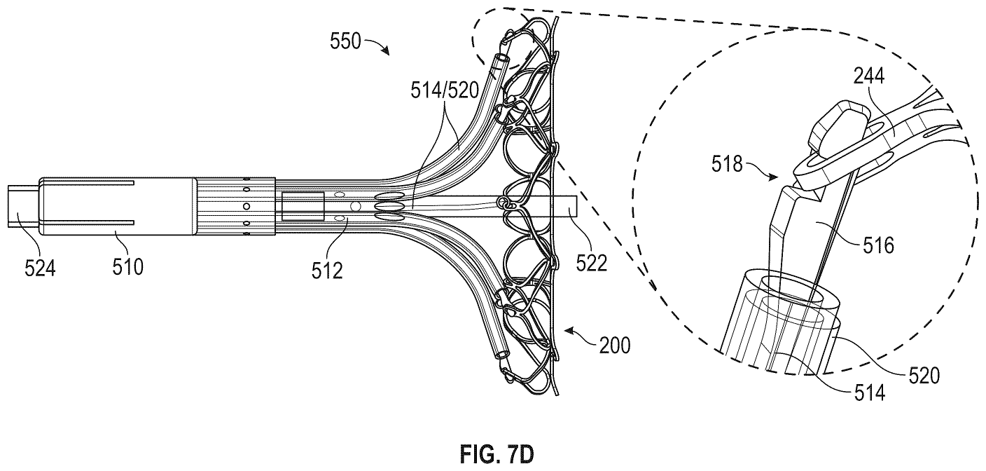

FIGS. 5B and 5C illustrate an embodiment of the implant loader 504 of the delivery system 500. As shown, prior to implantation, a loader luer 510 is connected to the proximal end of the sheath 506. The grasping system 550 may be used to grasp the occluding device 200 (e.g., at the outer petal tips 244) for delivery. The grasping system 550 is described further in FIGS. 6A-7D. In operation, the occluding device 200 in a compressed configuration (e.g., FIG. 5C) is connected to the grasping system 550 and the grasping system 550 is pushed/advanced through the sheath 506 until the occluding device 200 reaches the target destination within the patient's heart. While FIGS. 5A-5C, 6A-6B, and 7C-7D illustrate only the support structure 201 of the occluding device 200, it is recognized that the support structure 201 is displayed for illustrative purposes, and in operation, a complete occluding device 200 would be implanted in a patient.

b. Release System

FIGS. 6A and 6B illustrate a side view and a perspective view of the grasping system 550 respectively. The grasping system 550 (also referred to herein as a release system/release member) comprises a plurality of graspers 514 connected to a connector 512. Each individual grasper 514 is connected at a proximal end to the connector 512 and comprises a flag 516 at the distal end. The flags 516 may be connected to the outer petal tips 244 of the occluding device 200 for delivery and release of the occluding device 200. The graspers 514 may comprise thin projections extending distally from the connector 512. Like the support structure 201, the graspers 514 may be configured to expand and contract between a compressed tubular configuration for insertion through a patient's vasculature and an expanded or extended configuration in which the flags 516 extend radially outwards from a central portion (e.g., the central portion 206 of the occluding device 200). The flags 516 may comprise a wider portion than the graspers 514 with a cutout 518. The flags 516 and the cutouts 518 may be configured to interface with the outer petal tip 244 of the support structure 201. For example, a flag 516 may extend through the loop of the outer petal tip 244 such that the loop of the outer petal tip 244 is positioned within the cutout 518. In the expanded configuration, shown in FIGS. 6A and 6B, the connection between the flags 516 and outer petal tips 244 may be loose, such that minimal or no movement of the graspers 514 causes the flags 516 to disconnect from the outer petal tips 244, releasing the occluding device 200. To prevent the unintended release of the occluding device 200, each graspers 514 may be covered by a lock tube 520 as described in FIGS. 7A-7D.

In some embodiments, the grasping system 550 may comprise the same number of graspers 514 as the outer petal tips 244 of an occluding device 200 being used in an operation. For example, each grasper 514 may have a corresponding outer petal tip 244 to connect to. For example, the grasping system 550 may comprise 2, 4, 6, 8, 10, 15, 20, 25, 30, and/or the like graspers 514. In some embodiments, the graspers 514 may comprise a metal, a plastic, and/or the like. In some embodiments, the graspers 514 may comprise a shape-memory material, such as, for example, Nitinol. A shape-memory material may allow the graspers 514 to conform to the tubular configuration and self-expand once the graspers 514 are released from the dial end of the sheath 506.

FIG. 7A illustrates an embodiment of the graspers 514 and the connector 512 of the grasping system 550 positioned on a guidewire lumen 522. The grasping system 550 connected to the occluding device 200 may travel along the guidewire lumen 522 within the sheath 506 within a patient's vasculature prior to implantation.

FIG. 7B illustrates an embodiment of the grasping system 550 further including a plurality of lock tubes 520. In some embodiments, the lock tubes 520 may be connected to a release lumen 524 as described further below. The lock tubes 520 may comprise hollow tubes that extend distally away from the connector 512. As shown in FIG. 7C, the graspers 514 extend through the lock tubes 520. In some embodiments, including the embodiment illustrated, the lock tubes 520 may include cut outs (skives) 526 near the proximal end to allow the lock tubes 520 to be threaded onto the graspers 514 (i.e., individually attached). In this configuration, the lock tubes 520 are then able to be longer than the graspers 514 and can be effectively actuated as a result. In operation, before deployment of the occluding device 200, the lock tubes 520 extend over the flags 516 and the outer petal tips 244, which prevents the occluding device 200 from being released prematurely. Like the graspers 514, the lock tubes 520 may be configured to expand and contract between a compressed tubular configuration for insertion through the patient's vasculature and an expanded configuration in which the distal ends of the lock tubes 520 extend radially outwards from a central portion (e.g., the central portion 206 of the occluding device 200). In the compressed configuration, the plurality of lock tubes 520 may be in contact with each other and help to secure the occluding device 200 to the grasping system 550. In the expanded configuration, the lock tubes 520 extend with the graspers 514 until the occluding device 200 reaches a fully or almost fully expanded position.

The lock tubes 520 may comprise any suitable material that allows the lock tubes 520 to be compressed for delivery and readily expandable for deployment of the occluding device 200. In some embodiments, the lock tubes 520 may comprise a plastic, polymer, metal, and/or the like. In some cases, the lock tubes 520 may compromise a combination material such as a polymer covered coil, for example.

FIG. 7D illustrates the lock tube 520 and grasper 514 system where the lock tubes 520 have been retracted to expose the flags 516 of the graspers 514. The release lumen 524 may be retracted towards to the proximal end of the delivery system 500, which may cause the lock tubes 520 to also retract proximally to expose the flags 516. In some embodiments, the handle 502 may include an actuator (e.g., release button 501) that controls the release lumen 524. For example, the release button 501 may be configured to move from a lock position, where the lock tubes 520 cover the flags 516 of the graspers 514, to a release position, where the release lumen 524 and the lock tubes 520 are retracted proximally, causing the flags 516 to be exposed. Once the lock tubes 520 are retracted, the occluding device 200 may be released from the delivery system 500 automatically or with slight movement of the occluding device 200 against, for example, the partition 7 where the occluding device 200 has been deployed.

The combination of the graspers 514 and the lock tubes 520 as well as the outer petal tips 244 of the occluding device 200 may allow the occluding device 200 to be in a completely or almost completely expanded configuration prior to the actual deployment of the occluding device 200. This system may allow the operator (e.g., a physician) of the delivery system 500 to optimize the position of the occluding device 200 within a patient's heart before having to release the device. Further, the system may allow the operator to completely retract the expanded occluding device 200 back into the delivery system 500. For example, if the occluding device 200 is expanded in the non-optimal position or the something has gone wrong with the operation, the system allows the operator to retract the fully expanded occluding device 200 back into the delivery system 500 and optionally out of the patient. Once the operator is confident the occluding device 200 is secure in position (e.g., within the lumen 4 of the heart 103), the operator may retract the lock tubes 520 and allow the occluding device 200 to be released from the delivery system 500.

### V. Example Deployment

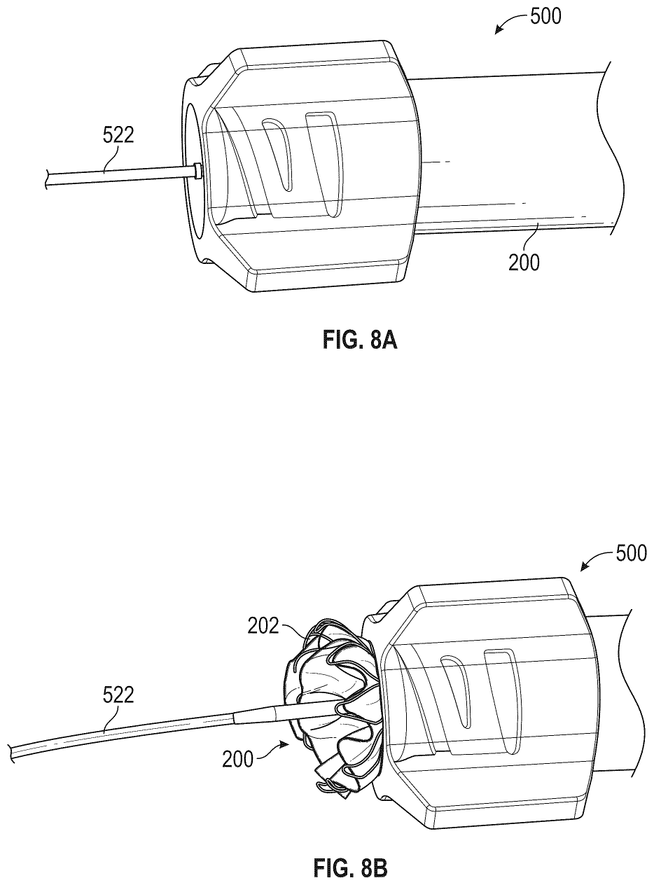

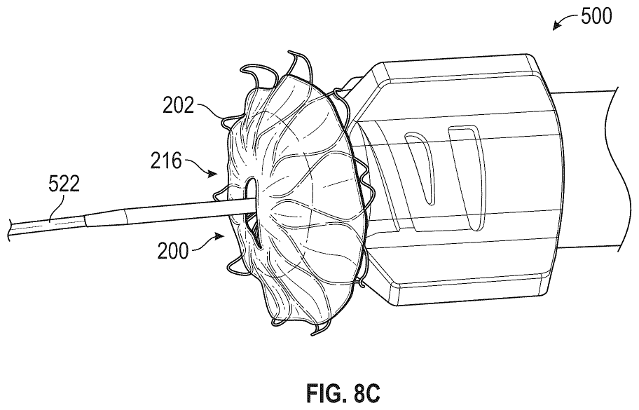

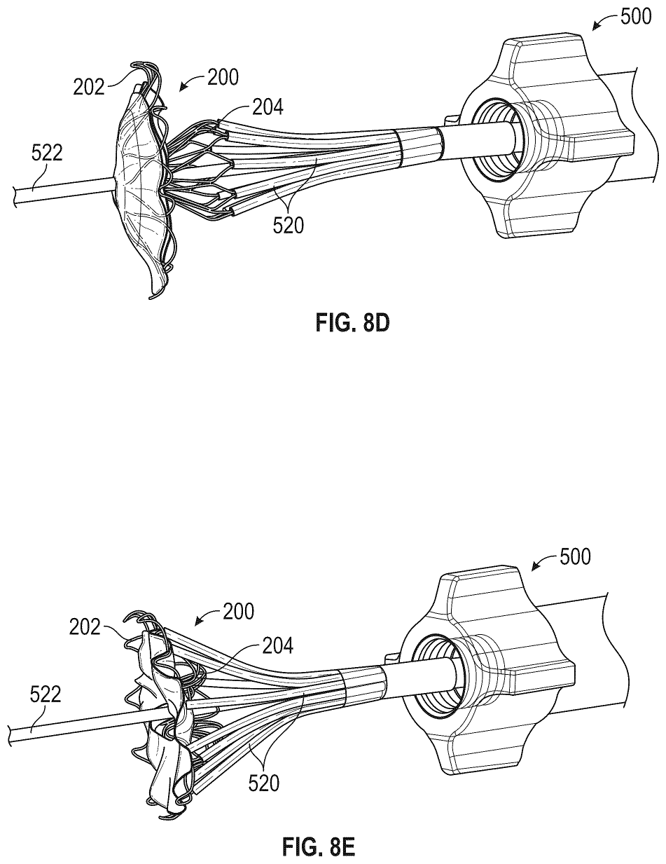

FIGS. 8A-8E illustrate an example deployment of an embodiment of an occluding device 200. FIGS. 8A-8E show the occluding device 200 expanding outside of handle 502 of the delivery system 500. However, it is recognized that in operation, the occluding device 200 would be attached to the delivery system 500, travel over-the-wire through the sheath 506 to the target location. The sheath 506 would be prepositioned across the atrial septal defect (the target location) from the right atrium to the left atrium. As described herein, the delivery system 500 permits the repositioning and retrieval of the occluding device 200, prior to final release, if necessary.

Generally, the occluding device 200 may be advanced to the heart using conventional transcatheter techniques via transfemoral venous approach. For example, a dilator (e.g., 12 Fr) and the delivery sheath 506 may be inserted into the femoral vein, advanced along the inferior vena cava and into the right atrium. Once the distal end of the delivery sheath 506 is across the ASD, the dilator may be removed. The occluding device 200, attached to the delivery system 500, may be delivered through the delivery sheath 506 over a guidewire (not shown), under fluoroscopic guidance. For example, after de-airing the delivery system 500 lumens and the implant loader 504, the occluding device 200 may be sheathed into the implant loader 504. The implant loader 504 may be attached to the prepositioned delivery sheath 506 such that the occluding device 200 can be advanced through the delivery sheath 506. The handle 502 may engage the implant loader 504, permitting stepwise deployment of the occluding device 200 as described below. The handle 502 may be used to control predictable positioning, deployment, and release of the occluding device 200 across the ASD using various mechanisms. In one example, a physician may rotate rotation knob 503 to retract the sheath 506 to deploy the occluding device 200 and the release button 501 to release the occluding device 200. For example, with the distal end of the sheath 506 across the ASD, the physician may rotate rotation knob 503 in a first direction a first amount to partially retract the sheath 506 and deploy the first anchoring portion 202 in the left atrium 102. Once successful deployment of the first anchoring portion 202 is confirmed, the physician may retract the delivery system 500 proximally until the first anchoring portion 202 contacts the septum (e.g., partition 7). While maintaining the position of the delivery system 500, the physician may then continue to rotate the rotation knob 503 in the first direction a second amount to fully retract the sheath 506 to deploy the second anchoring portion 204 in the right atrium 101. At this point, the physician may confirm the occluding device 200 is correctly deployed and/or positioned. If the occluding device 200 is not correctly deployed or positioned, the physician may partially or fully rotate the rotation knob 503 in a second direction to retract and re-sheath the occluding device 200 into the sheath 506. At this point, the delivery system 500 can be repositioned for another deployment attempt. Once successful deployment in confirmed, the physician may actuate the release button 501 (e.g., move the release button 501 proximally) to retract the lock tubes 520 and permit detachment of the occluding device 200 from the grasping system 550. In some embodiments, the handle 502 may include one or more visual indicators that indicate when the rotation knob 503 has rotated the first amount and the first anchoring portion 202 is deployed and when the rotation knob 503 has rotated the second amount and the second anchoring portion 204 is deployed.

FIG. 8A illustrates the occluding device 200 in a contracted configuration within the delivery system 500. As shown, the guidewire lumen 522 extends distally from the delivery system 500.

FIG. 8B illustrates the occluding device 200 beginning to exit the delivery system 500. The occluding device 200 is positioned such that the first anchoring portion 202 exits the delivery system 500 before the second anchoring portion 204. As shown, as the occluding device 200 exits the delivery system 500, the first anchoring portion 202 immediately begins to expand to an extended configuration. In an operation, the delivery system 500 would be positioned such that the first anchoring portion 202 expands on one side of the partition 7, such as, for example, within the left atrium 102 of the heart 103.

FIG. 8C illustrates the occluding device 200 where the first anchoring portion 202 has fully exited the delivery system 500. In the illustrated configuration, the outer diameter D2 of the occluding device 200 would generally be larger than the target lumen 4 of the heart 103 such that the first anchoring portion 202 would have to be retracted back within the delivery system 500 to be retracted through the lumen 4.

FIG. 8D illustrates the occluding device 200 where both the first anchoring portion 202 and the second anchoring portion 204 have fully exited the delivery system 500. As shown, the second anchoring portion 204 is not fully expanded and is still connected to the flags 516 of the graspers 514 within the lock tubes 520. In some embodiments, the second anchoring portion 204 may not fully expand until the first anchoring portion 202 is retracted to contact the partition 7 of the heart 103. For example, once the first anchoring portion 202 is expanded within the left atrium 102, the first anchoring portion 202 may be retracted such that the petals 228 contact the atrial partition 100. When the atrial partition 100 exerts a force on the petals 228, the first anchoring portion 202 may be partially inverted (e.g., as shown in FIG. 8E) which may cause the second anchoring portion 204 to expand to a fully expanded position, compressing the partition between the first anchoring portion 202 and the second anchoring portion 204. In some embodiments, the first anchoring portion 202 may be released from the delivery system 500 in a fully operational state and may not need to be retracted against a partition to be inverted.

With continued reference to FIG. 8E, once the second anchoring portion 204 is fully expanded, in operation, the partition 7 would be compressed between the first anchoring portion 202 and the second anchoring portion 204. At this point, the occluding device 200 would still be connected to the delivery system 500. Once the operator was confident the occluding device 200 was in the correct position (e.g., with the lumen 208 positioned inside lumen 4 with the partition 7 compressed between the first and second anchoring portions 202, 204), the operator may retract the release lumen 524, causing the lock tubes 520 to expose the flags 516 and the outer petal tips 244 of the second anchoring portion 204. This action may cause the occluding device 200 to be released or the operator may be required to gently move the occluding device 200 until the outer petal tips 244 are separated from the flags 516. After this point, the delivery system 500 may be retracted from the patient and the operation may be complete.

### VI. Occluding Device

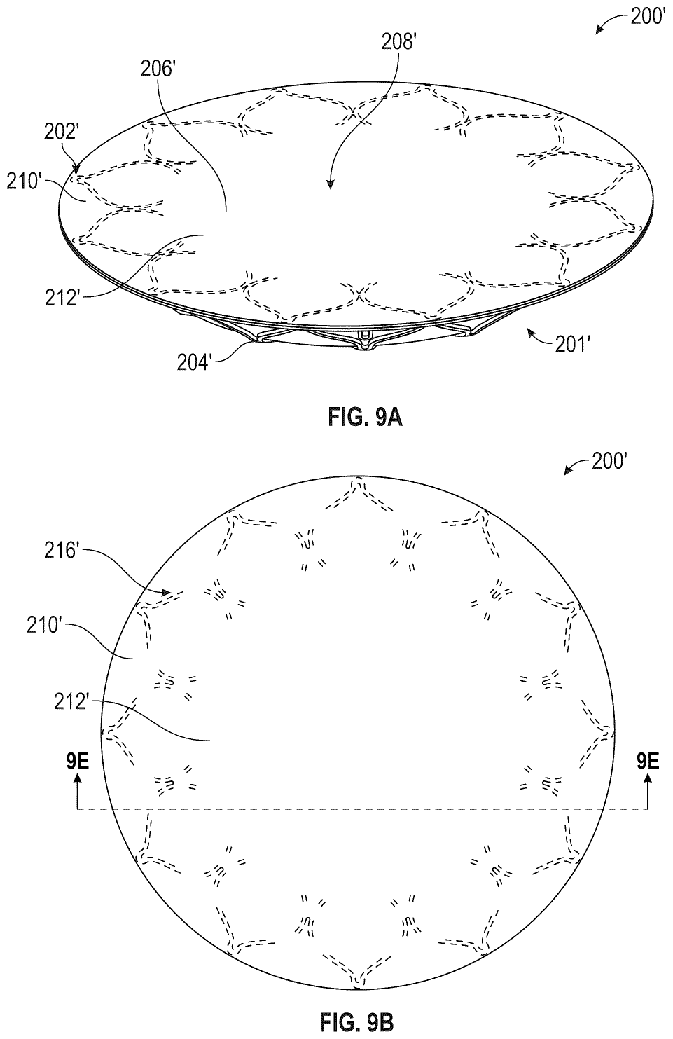

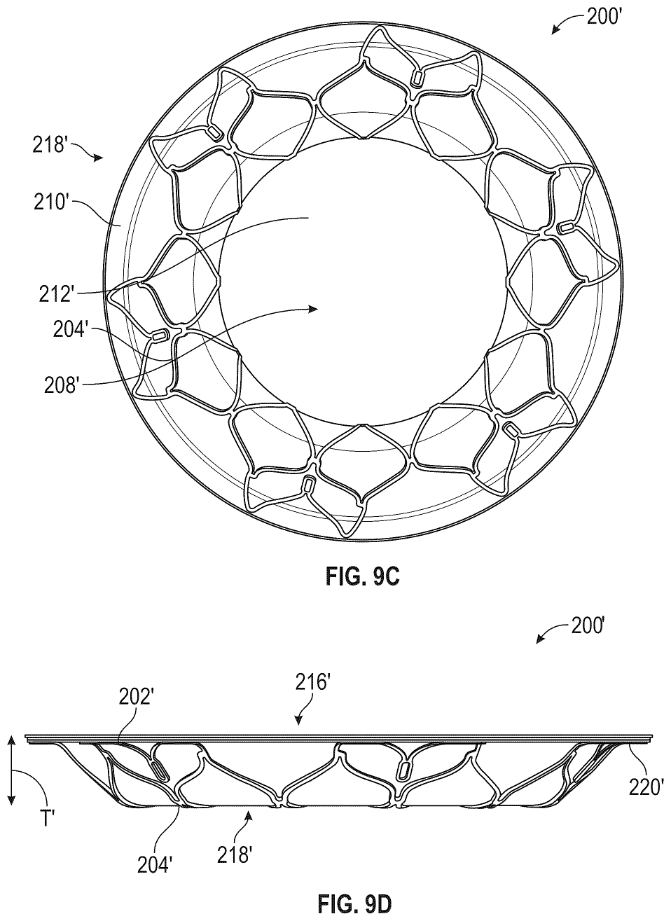

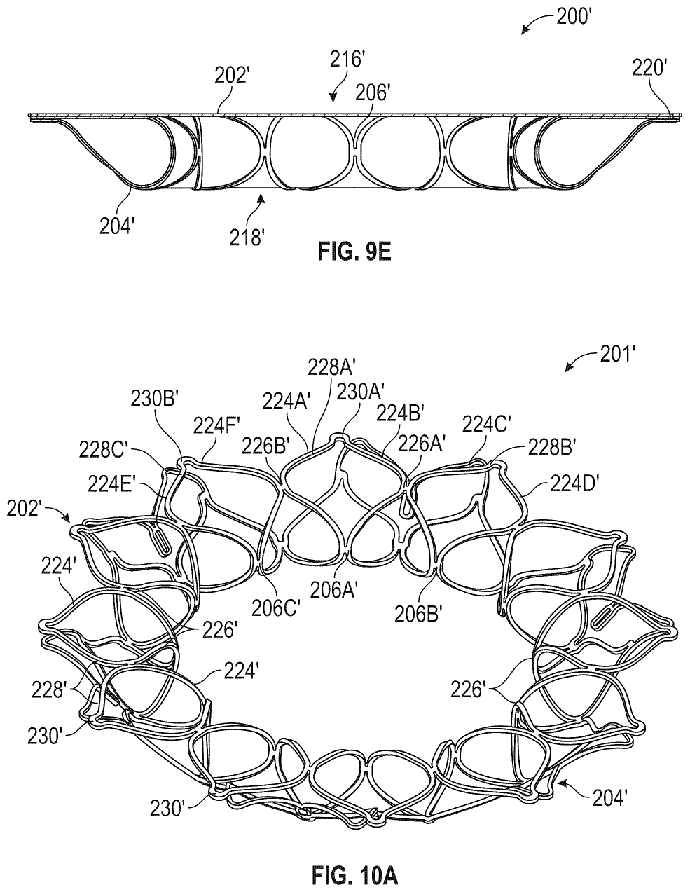

FIGS. 9A-9E illustrate an occluding device 200′ in an expanded configuration. FIG. 9A illustrates a perspective view of the occluding device 200′, FIG. 9B illustrates a left atrial side view of the occluding device 200′, FIG. 9C illustrates a right atrial side view of the occluding device 200′, FIG. 9D illustrates a side view of the occluding device 200′, and FIG. 9E illustrates a section view of the occluding device 200′. The occluding device 200′ may include all of the same components as the occluding device 200 and may function in a similar or identical manner to the occluding device 200. Components of the occluding device 200′ that share the same function and properties as the occluding device 200 are labeled with the same reference number with a prime designation (e.g., support structure 201′).

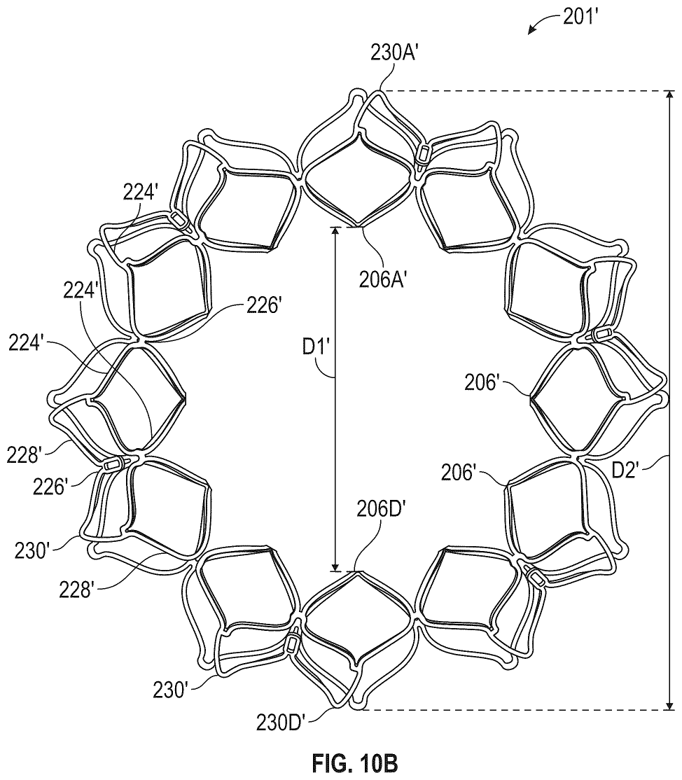

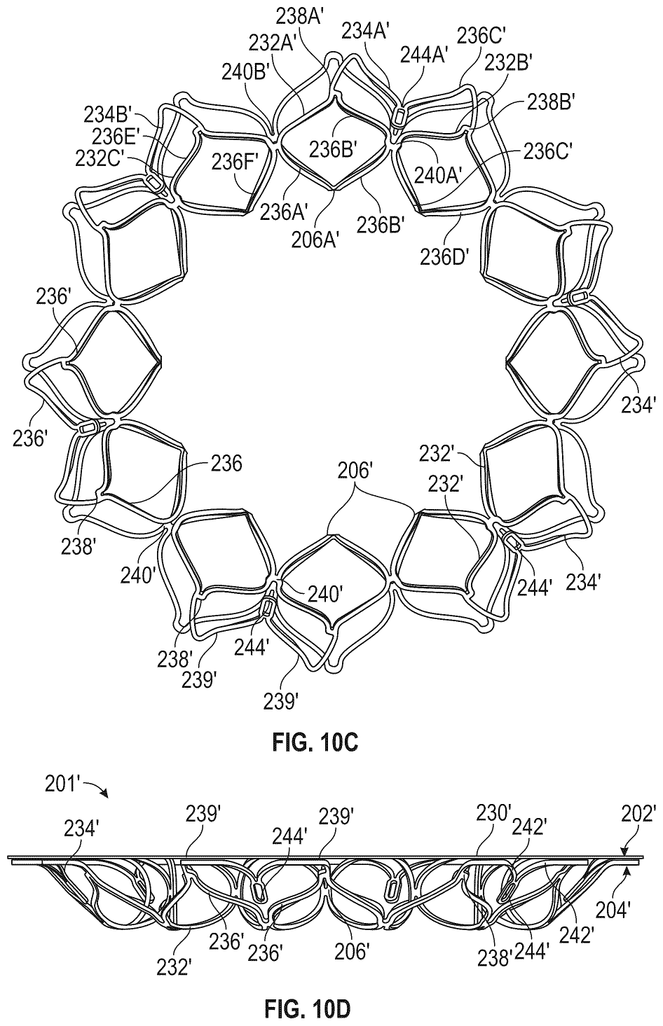

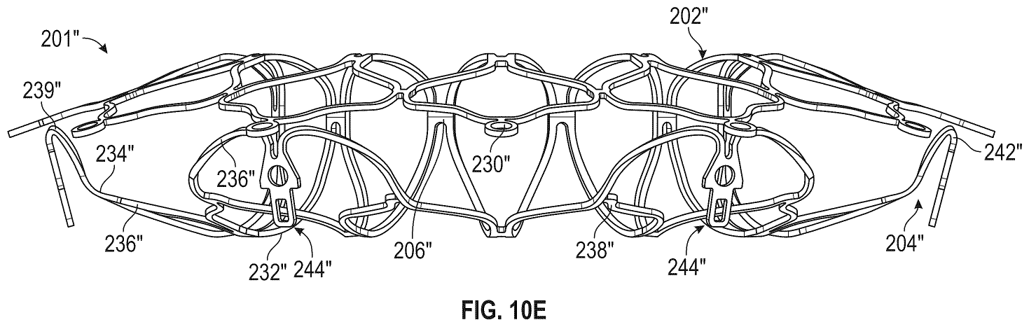

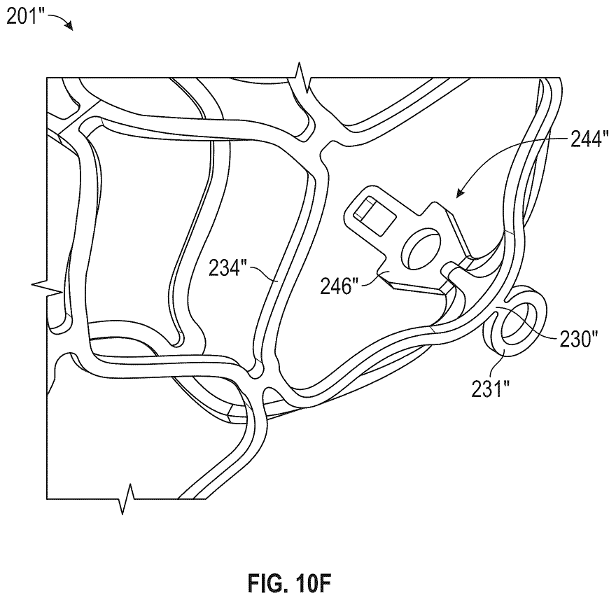

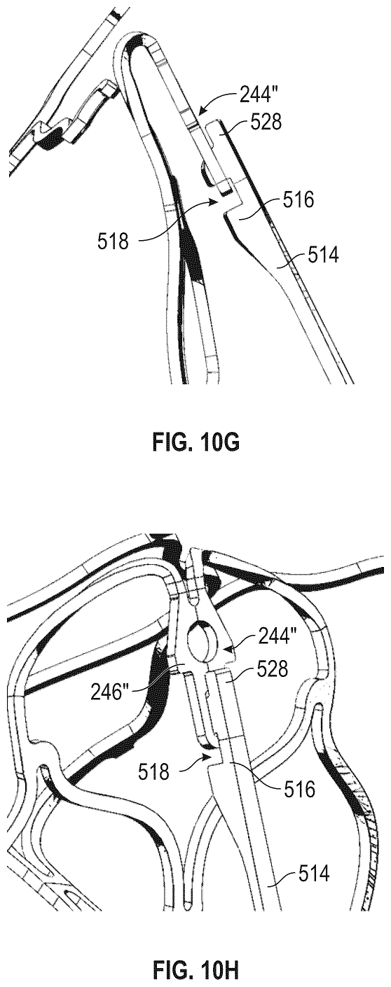

FIGS. 10A-10D illustrate the frame/support structure 201′ of the occluding device 200′. FIG. 10A illustrates a perspective view of the frame 201′, FIG. 10B illustrates a left atrial side view of the frame 201′, FIG. 10C illustrates a right atrial side view of the frame 201′, and FIG. 10D illustrates a side view of the frame 201′.

As seen most clearly in FIGS. 9E and 10D, one of the differences between support structure 201′ of the occluding device 200′ and the support structure 201 of the occluding device 200, is the that the support structure 201′ is shaped to further eliminate discontinuities of the occluding device 200′ once implanted. For example, the outer edge 220′ of the second anchoring portion 204′ does not include an inflection portion (e.g., see outer edges 222 in FIG. 2E). Instead, the outer edge 220′ is substantially parallel to the occlusion membrane 212′, when the occlusion membrane 212′ is coupled to the first anchoring portion 202′ (i.e., as opposed to the example where the occlusion membrane 212′ is coupled to the central portion 206′). As a result, the outer edge 220′ of the first anchoring portion 202′ and the left atrial side 216′ of the occluding device 200′ lies substantially flat on the left atrial 102 side of the septum when the occluding device 200′ is implanted.

Similarly, as seen most clearly in FIGS. 10C and 10D, the outer petals 234′ of the second anchoring portion 204′ may be shaped differently that the outer petals 234 of the second anchoring portion 204 in the occluding device 200. For example, the struts 236′ of the outer petals 234′ may extend outwardly from the inner petal tips 238′ in a similar manner as the struts 236 of the outer petals 234 in the second anchoring portion 204 in the occluding device 200. However, in the second anchoring portion 204′, the struts 236′ of the outer petals 234′ extend to an outer petal edge 239′, which is at a maximum outside radial location for the outer petals 234′. At the outer petal edges 239′, the struts 236′ inflect back towards the outer petal tips 244′ in a direction downwardly towards the right atrial side 218′ and inwardly towards the central portion 206′ of the support structure 201′. As a result of this inflection, the outer petal tips 244′ are positioned within the outer petals 234′, and the outer petal edges 239′ represent the outer edges of the outer petals 234. Additionally, the struts 236′ between the outer petal edges 239′ and the outer petal tips 244′ are at a similar angle relative to the horizontal as the struts 236′ between inner petal connectors 240′ and the inner petal tips 238′. As such, the outer petal tips 244′ and the second anchoring portion 204′ as a whole lies substantially flat on the right atrial 102 side of the septum when the occluding device 200′ is implanted.

FIGS. 10E and 10F illustrate an implementation of an additional frame/support structure 201″ that can be used as part of the occluding device 200 or occluding device 200′. FIG. 10E shows a side view of the frame 201″ and FIG. 10F shows a detailed view of a portion of the frame 201″. The frame 201″ may include all of the same components as the frame 201 of the occluding device 200 and the frame 201′ of the occluding device 200′ and may function in a similar or identical manner to the frames of the occluding devices 200, 200′. Components of the frame 201″ that share the same function and properties as the frames 201, 201′ are labeled with the same reference number with a double prime designation (e.g., support structure 201″).

The frame 201″ differs from the frames 201, 201′ primarily in the structure of the frame 201″ at the petal tips. For example, one or more of the petal tip 230″ on the first anchoring portion 202″ can include marker tips 231″. The marker tips 231″ can extend from the petal tips 230″. In the illustrated example, the marker tips 231″ extend inwardly towards the lumen 208″. In some cases, the marker tips 231″ can be radio opaque, such that a physician can identify an outer boundary of the frame 201″ once implanted. The marker tips 231″ can be designed as atraumatic tips to minimize trauma to the patient during implantation. For example, the marker tips 231″ can be generally circular shaped. In some implementations, each petal tip 230″ may include a marker tip 231″. In other implementations, alternating petal tips 230″ may include a marker tip 231″.

The outer petal tips 244″ of the frame 201″ may include shoulder stops 246″. The shoulder stops 246″ may be portions of the outer petal tips 244″ that are located inwardly of the eyelets of the outer petal tips 244″ and have a greater width than the width of the eyelets. The shoulder stops 246″ can be used to prevent or limit movement of the lock tubes 520 over the rest of the frame 201″. For example, when coupled to the delivery system 500, the outer petal tips 244″ may be connected to the flags 516 of the graspers 514 via the eyelets in the outer petal tips 244″. The lock tubes 520 can extend over the eyelets and the flags 516, ensuring that the frame 201″ is locked to the delivery system 500, and the shoulder stops 246″ can limit the extension of the lock tubes 520 over the outer petal tips 244″. Additionally, as shown in FIG. 10E, the struts 236″ extending towards the outer petal tips 244″ can be folded in plane with the second anchoring portion 204 and can be bent outward for improved attachment to the delivery system 500.

FIGS. 10G and 10H illustrate detailed views of an outer petal tip 244″ of the support structure 201″ engaged with a grasper 514 of the delivery system 500. As shown in FIGS. 10G and 10H, to ensure disconnection after removal of the lock tubes 520, the flags 516 may each include an extension tab 528. The extension tab 528 may be a portion of material extending from the flags 516 that is greater than the length of the eyelet of the outer petal tip 244. The extension tab 528 can prevent the flags 516 from protruding through the outer petal tips 244. For example, because of the inclusion of the extension tab 528, the flags 516 cannot extend through the eyelets of the outer petal tips 244″. Additionally, in some examples, a portion of the flags 516 proximal to the cutout 518 may have a greater thickness than the length/diameter of the eyelets of the outer petal tips 244, such that this portion of the cutout 518 cannot extend through the eyelets. This arrangement can prevent the flags 516 and the remainder for the graspers 514 from completely extending through the petal tip eyelets 244.

### VII. Occluding Device

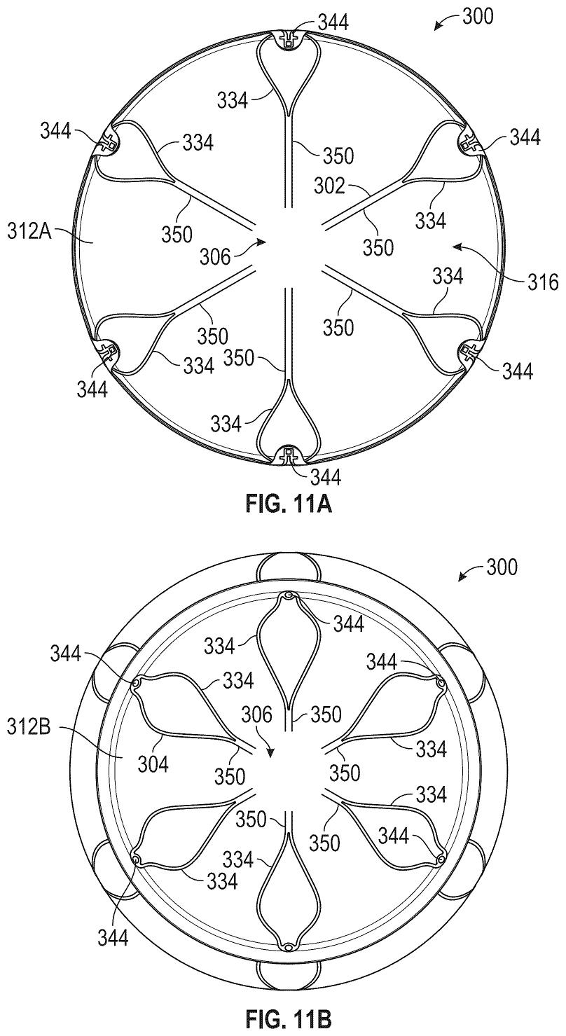

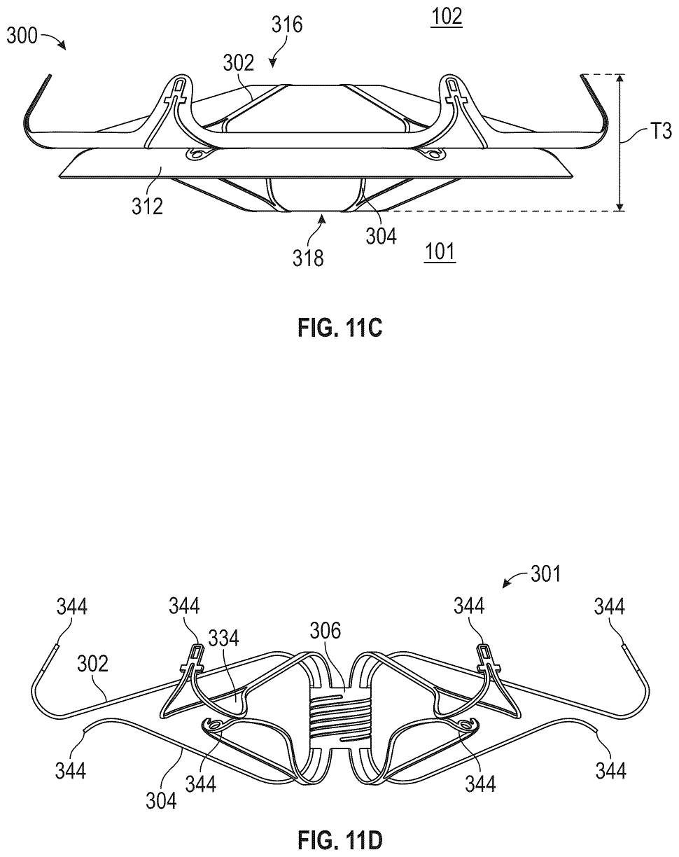

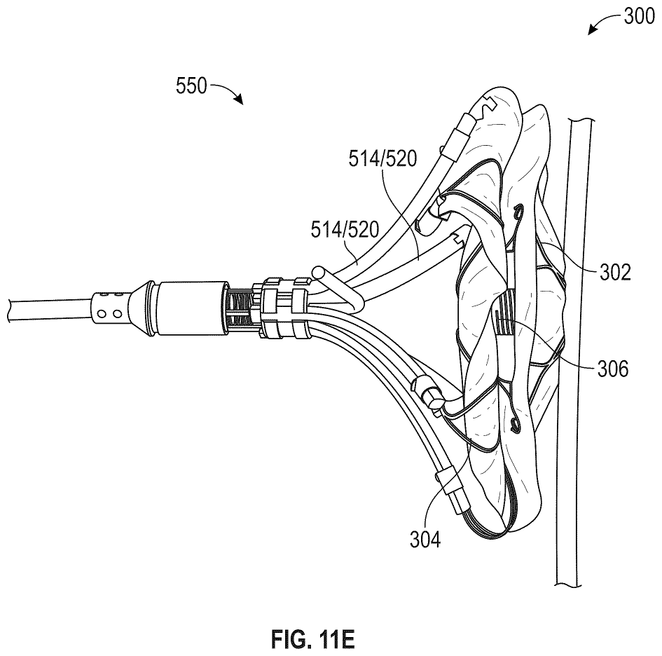

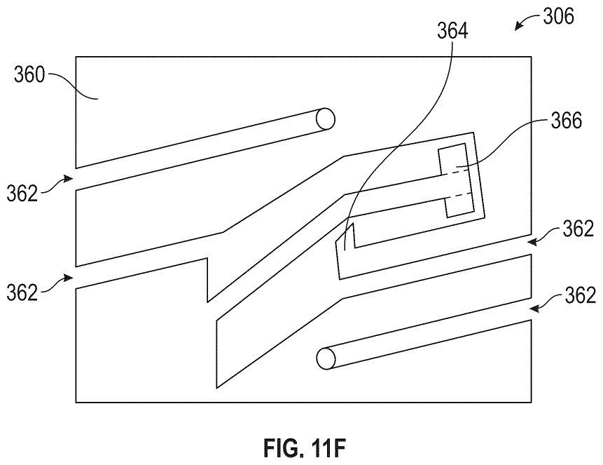

FIGS. 11A-11E illustrate various views of an occluding device 300. The occluding device 300 can include some or all of the structures and functionalities as the occluding device 200 as shown and described in relation to at least FIG. 2A-4C, with the differences noted below. Thus, reference numerals used to designate various features or components of the occluding device 200 are identical to those used for identifying the corresponding features of the components of the occluding device 300, except that the numerical identifiers for the occluding device 300 include a “3” instead of a “2”.

FIGS. 11A and 11B illustrate a left atrial side view and a right atrial side view respectively of the occluding device 300. The occluding device 300 differs from the occluding device 200 primarily in the configuration of the support structure 301 and the central portion 306. In some implementations, the occluding device 300 may provide particular benefits when implemented to occlude a PFO defect. However, the occluding device 300 can also be used for treatment of other defects (e.g., ASD). The support structure 301 can include a first anchoring portion 302 and a second anchoring portion 304. In some implementations, the first anchoring portion 302 can be identical to the second anchoring portion 304. The anchoring portions 302, 304 can include a plurality of struts 350 that extend radially outward from the central portion 306. The plurality of struts 350 can extend towards outer petals 334. The outer petals 334 can include outer petal tips 344. The outer petal tips 344 can be configured to be interface with/coupled to graspers 514 of the delivery system 500 (e.g., via the flags 516). For example, the outer petal tips 344 can be or can include eyelets. In some implementation, all or a portion of the outer petals 334 may be radiopaque.

The support structure 301 can be covered by a membrane. The first anchoring portion 302 can be covered by a first membrane 312A and the second anchoring portion 304 can be covered by a second membrane 312B. In some implementations, a single membrane 312 may cover both the first anchoring portion 302 and the second anchoring portion 304. The membranes 312A, 312B can have the same or identical structure and features as the frame covering membrane 210 and the occlusion membrane 212 of the occluding device 200. The occluding device 300 may include a smaller central portion 306 and lumen 308 compared to the occluding device 200. The membranes 312A, 312B can extend over the lumen 308 on both sides of the occluding device 300 (e.g., on the first anchoring portion 202 and second anchoring portion 204). Like the occluding device 200, the occluding device 300 can be re-crossable once implanted. A physician may re-cross the occluding device 300 through the lumen 308, and/or between a pair of struts 350. In some embodiments, the membranes 312A, 312B covering may only extend partially outwards on either the left atrial disc 301 or right atrial disc 302 or both. In some implementations, the occluding device 300 can be used as a shunt. For example, the occluding device 300 can include one or more permanent openings (e.g., holes, slits, and/or the like) that extend through the occluding device 300 and can be used to exchange fluid across the occluding device 300.

FIG. 11C illustrates a side view of the occluding device 300 implanted in the partition 7 of the heart. As shown, the implanted occluding device 300 (pre-endothelization) has a similar thickness to the partition 7. As noted herein, in the heart, the septum may have a thickness of approximately 1 mm-5 mm (depending on the patient and the position of the defect) where the occluding device 300 is to be implanted. As such, it may be desirable to minimize a thickness T3 of the occluding device 300 to reduce the extension of the occluding device 200 into the left and right atriums 102, 101. The compliant nature of the occluding device 300 may be attributed in part to the thickness T3 of the occluding device 300. In some examples, the occluding device 300 may have a thickness T3 of between, 1 mm to 5 mm, 2 mm to 5 mm (e.g., 1 mm to 5 mm, 2 mm to 5 mm, 2.5 mm to 4.5 mm, 3 mm to 4 mm, values between the foregoing, etc.). In some cases, it may be desirable for the occluding device 300 to be 5 mm thick or less. The occluding device 300 may be substantially thinner than commercial occluding devices, which can have a thickness of 5 mm-10 mm or greater. In some implementations, the occluding device 300 can include a central connector that can expand and contract, giving the occluding device 300 a variable thickness, as described with reference to FIG. 11E.

FIG. 11D shows a side view of the support structure 301 of the occluding device 300 (e.g., with the single membrane(s) 312 removed. FIG. 11E illustrates a side view of the occluding device 300 in an expanded but not-implanted state coupled to the grasping system 550 of the delivery system 500. The occluding device 300 can include a central portion 306 that extends between the first anchoring portion 302 and the second anchoring portion 304. The central portion 306 can be used to couple the first anchoring portion 302 to the second anchoring portion 304. In some implementations, the central portion 306 can be or can include a resilient member. For example, the central portion 306 can be an expandable connection between the first anchoring portion 302 and the second anchoring portion 304. In one example, the central portion 306 can be a spring. The resilient central portion 306 can allow the first anchoring portion 302 and the second anchoring portion 304 to apply a compressive force to the partition 7 when the occluding device 300 is implanted in the heart. Because the central portion 306 is expandable, the occluding device 300 can accommodate different tunnel lengths through the septum. For example, because the septum has a varying thickness, the length of a defect (e.g., a tunnel) can vary. As the central portion 306 is expandable, the same occluding device 300 can be used for varying defect tunnel lengths, while still providing a similar compression on the septum and occlusion of the defect. In some implementations, the central portion 306 may be a triple start spring that is configured to stretch under low force. In one non-limiting example, the central portion 306 may have approximately 2-10 mm deflection at 4.5 N. The central portion 306 can also be configured to bend. As such, the first anchoring portion 302 and the second anchoring portion 304 may not be parallel planes once implanted. A bendable central portion 306 can provide a benefit of allowing the first anchoring portion 302 and second anchoring portion 304 to rest in non-parallel positions once implanted to better conform to the unique geometry on an individual patient's heart. While the central portion 306 is illustrated as exposed in FIG. 11C, in some implementations, the central portion 306 may be covered by a membrane similar to the membranes 312A, 312B. Additionally, the spring 306 can be designed with a variable width helical coil to achieve a constant force over a range of extensions. In one such example, the spring 306 can be fabricated using a helical cut in a tube. The cut pattern can have a variable thickness to achieve a constant force. In another example, the helical cut out can be shape-set inward (i.e., hourglass-shaped) or alternatively outwards (i.e. barrel-shaped) to achieve the desired spring performance. Further, specific limiting elements can be included between the coils of the spring 306 to prevent over-extension. For example, the spring 306 can be covered with an elastic polymer. In another example, sliding elements can be introduced into the structure of the helix. The sliding elements can be free to move but limit the extension of the helix past a set point.

FIG. 11F illustrates a schematic side view of an example central portion 306 formed as a spring that can be included in the occluding device 300. As shown, the central portion 306 can be formed from a helical coil 360. There can be gaps 362 between the helical coil 360. In some examples, the helical coil 360 can include one or more sliding element 364 and one or more stopping elements 366. The sliding element 364 can be portions of the helical coil 360 that extend into a gap 362 and can move within the gap 362 as the spring 306 extends and contracts. For example, as shown in FIG. 11F, extension of the spring 306 would cause the sliding element 364 to move from left to right as illustrated and compression of the spring 306 would cause the sliding element 364 to move from right to left as illustrated. The stopping elements 366 can be portions of the helical coil 360 that extend into a gap 362 that includes a sliding element 364 and that are configured to limit the extension of the helical coil 360 past a set point. For example, extension of the helical coil 360 can continue until the sliding element 364 contacts the stopping elements 366, preventing further movement of the sliding element 364 in that direction and preventing further extension of the helical coil 360.

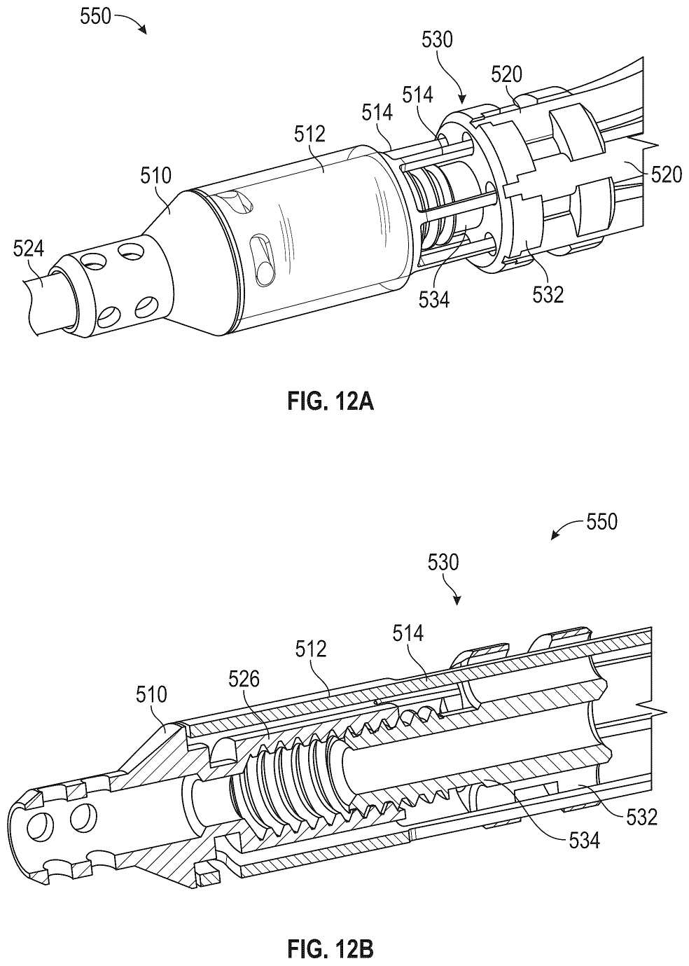

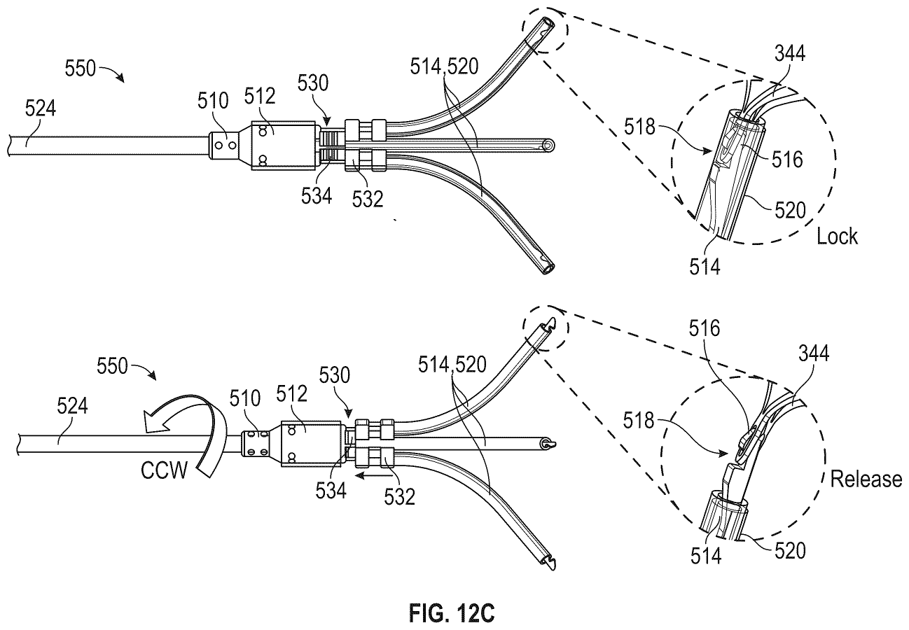

FIGS. 12A-12C illustrate additional and/or alternative features and components that can be implemented into the delivery system 500. The delivery system 500 can be used to allow the occluding device 300 (or the occluding device 200, or occluding device 200′) to be deployed by converting rotational motion (e.g., using a release knob) on the handle 502 to linear motion to retract the lock tubes 520. For example, a physician may rotate a release knob on the handle 502 to deploy the occluding device 200, 200′, 300, without causing corresponding rotation of the occluding device 200, 200′, 300.

FIG. 12A shows a partial view of the distal end of the delivery system 500, including the grasping system 550. FIG. 12B shows a section view of the distal end of the delivery system 500. The grasping system 550 can include a lock tube holder 530. The lock tube holder 530 can include a connection portion 532 and a shaft portion 534. The connection portion 532 can be coupled the shaft portion 534. The connection portion 532 is configured to hold the lock tubes 520 in fixed locations relative to each other. The connection portion 532 can allow the graspers 514 to extend through the lock tubes 520 and the connection portion 532. The shaft portion 534 can extend proximally from the connection portion 532. The shaft portion 534 can include threads on at least a portion of its external surface. The connection portion 532 can be distal to the loader luer 510 and the shaft portion 534 can extend at least partially into the loader luer 510. In some implementations, including as illustrated, the connection portion 532 and the shaft portion 534 can be hollow, which can allow the lock tube holder 530 to travel over the guidewire lumen 522. The threaded shaft portion 534 can engage with an internally threaded cylinder 526 of the release lumen 524. The internally threaded cylinder 526 can be coupled to a distal end of the release lumen 524 and can be positioned within the connector 512. The internally threaded cylinder 526 can be configured to rotate within the connector 512 while being in a fixed position axially such that the internally threaded cylinder 526 cannot move in the distal and proximal directions. As such, rotation of the release lumen 524 (e.g., via a release knob) causes corresponding rotation of the internally threaded cylinder 526. As the internally threaded cylinder 526 rotates, the threads of the internally threaded cylinder 526 engage the threads of the shaft portion 534, causing the lock tube holder 530 to move proximally or distally, depending on the direction of rotation.

FIG. 12C illustrates a side view of the grasping system 550 in a locked and unlocked position. In one example, counterclockwise rotation of the release knob causes proximal movement of the lock tube holder 530. The connector 512 can be fixed relative to the loader luer 510. As such, as the lock tube holder 530 moves proximally, the lock tubes 520 retract, exposing the flags 516 of the graspers 514. With the flags 516 exposed, the outer petal tips 344 of the occluding device 300 are also released from the lock tubes 520, and the occluding device 300 can be deployed.

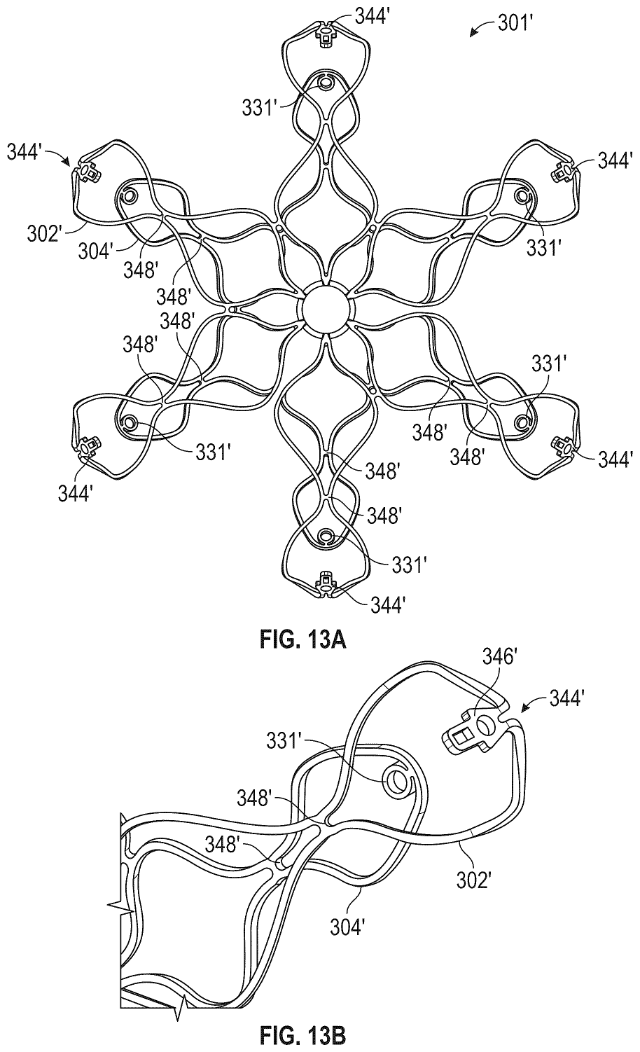

FIGS. 13A and 13B illustrate an implementation of an additional frame/support structure 301′ that can be used as part of the occluding device 300. FIG. 13A shows a side view of the frame 301′ and FIG. 13B shows a detailed view of a portion of the frame 301′. The frame 301′ may include all of the same components as the frame 301 of the occluding device 300 and may function in a similar or identical manner to the frame of the occluding device 300. Components of the frame 301′ that share the same function and properties as the frame 301 are labeled with the same reference number with a prime designation (e.g., support structure 201″).

The frame 301′ differs from the frame 301′ primarily in the structure of the frame 301′ at the petal tips. For example, one or more of the outer petal tips 344′ on the first anchoring portion 302′ or the second anchoring portion 304′ can include marker tips 331′. The marker tips 331′ can extend from the outer petal tips 344′. In the illustrated example, the marker tips 331′ extend inwardly towards the central portion 306′. In some cases, the marker tips 331′ can be radio opaque, such that a physician can identify an outer boundary of the frame 301′ once implanted. The marker tips 331′ can be designed as atraumatic tips to minimize trauma to the patient during implantation. For example, the marker tips 331′ can be generally circular shaped. In some implementations, each outer petal 334′ of one or both of the first or second anchoring portions 302′, 304′ may include a marker tip 331′. In other implementations, alternating outer petal 334′ in one or both of the first or second anchoring portions 302′, 304′ may include a marker tip 331′.

In some cases, the outer petal tips 344′ of the frame 301′ may include shoulder stops 346′. The outer petal tips 344′ with the shoulder stops 346′ may be on one of the first anchoring portion 302′ and the second anchoring portions 304′ while the outer petal tips 344′ with the marker tips 331′ may be on the opposite anchoring portion 302′, 304′. The shoulder stops 346′ may be portions of the outer petal tips 344′ that are located inwardly of the eyelets of the outer petal tips 344′ and have a greater width than the width of the eyelets. The shoulder stops 346′ can be used to prevent or limit movement of the lock tubes 520 over the rest of the frame 301′. For example, when coupled to the delivery system 500, the outer petal tips 344′ may be connected to the flags 516 of the graspers 514 via the eyelets in the outer petal tips 344′. The lock tubes 520 can extend over the eyelets and the flags 516, ensuring that the frame 301′ is locked to the delivery system 500, and the shoulder stops 346′ can limit the extension of the lock tubes 520 over the outer petal tips 344′. Additionally, as shown in FIG. 13B, the struts of the outer petal 334′ extending towards the outer petal tips 344′ can be folded in plane with the second anchoring portion 304 and can be bent outward for improved attachment to the delivery system 500.

Referring back to FIG. 13A, the struts 350′ of the frame 301′ may include one or more junctions 348′. The junctions 348′ can be sections of the frame 301′ where a pair of struts 350′ meet. The junctions 348′ can form one or more additional petals in the anchoring portions 302, 304. For example, each junction 348′ can define an intersection point of the outer petal 334′ and an inner petal 332′. Including junctions 348′ in the frame 301′ can increase the axial clamping force provided by the occluding device 300 once implanted.

### VIII. Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub- combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

### IX. Example Clauses

Examples of implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.

Clause 1. An interseptal occluding device comprising: a support structure comprising a first anchoring portion and an opposite second anchoring portion, a lumen extending through a center of the first anchoring portion and a center second anchoring portion, wherein the support structure is configured to contract and expand between a compressed tubular configuration for insertion through a patient's vasculature, and an expanded configuration, in which the first and second anchoring portions extend radially outwards from the lumen; and a membrane coupled to the first anchoring portion, the membrane configured to occlude a majority of the lumen when the support structure is expanded, the membrane configured to promote tissue growth at least across the membrane.

Clause 2. The interseptal occluding device of Clause 1, wherein the membrane comprises an elastic material.

Clause 3. The interseptal occluding device of Clause 1 or 2, wherein the membrane includes a plurality of perforations.

Clause 4. The interseptal occluding device of any of Clauses 1-3, wherein the membrane comprises a radiopaque material.

Clause 5. The interseptal occluding device of any of Clauses 1-4, further comprising a second membrane, wherein the second membrane extends over the first anchoring portion and the second anchoring portion without occluding the lumen when the support structure is expanded.

Clause 6. The interseptal occluding device of any of Clauses 1-5, wherein the first and second anchoring portions comprise a plurality of interconnected struts.

Clause 7. The interseptal occluding device of any of Clauses 1-6, wherein the first and second anchoring portions comprise a shape-memory material.

Clause 8. The interseptal occluding device of any of Clauses 1-7, wherein the second anchoring portion comprises a plurality of loops, wherein the plurality of loops is configured to releasably couple the interseptal occluding device to a delivery device.

Clause 9. The interseptal occluding device of any of Clause 1-8, wherein the first anchoring portion and the second anchoring portion are configured to compress a partition of a heart therebetween.

Clause 10. The interseptal occluding device of Clause 9, wherein the lumen is configured to be aligned with an opening in the partition.

Clause 11. The interseptal occluding device of Clause 9 or 10, wherein the membrane is configured to allow a medical instrument inserted in a first compartment of the heart to pass through the membrane and a tissue layer formed on the membrane through the lumen into a second compartment of the heart, wherein the partition divides the first and second compartments of the heart.

Clause 12. The interseptal occluding device of Clause 1-11, wherein the membrane forms a continuous surface.

Clause 13. The interseptal occluding device of Clause 5-12, wherein the second membrane prevents the support structure from directly contacting a patient's blood when the interseptal occluding device is implanted in the patient.

Clause 14. A method, comprising: inserting a delivery system into a patient's vasculature, the delivery system comprising a release member and a sheath, the sheath containing an interseptal occluding device in a compressed configuration, the interseptal occluding device comprising a first anchoring portion and an opposite second anchoring portion, the second anchoring portion coupled to the release member; advancing a distal end of the sheath at least partially across a partition in the patient's heart; advancing the interseptal occluding device within the sheath such that the first anchoring portion expands in a first compartment on a first side of the partition; advancing the interseptal occluding device outside of the sheath such that the second anchoring portion expands in a second compartment on a second side of the partition, the second side opposite the first side, wherein the second anchoring portion remains coupled to the release member in an expanded configuration; and releasing the interseptal occluding device from the delivery system.

Clause 15. The method of Clause 14, wherein a lumen extends through a center of the first anchoring portion and a center second anchoring portion, the lumen delimiting an opening in the partition.

Clause 16. The method of Clause 15, wherein the interseptal occluding device further comprises a membrane coupled to the first anchoring portion, the membrane configured to occlude a majority of the lumen.

Clause 17. The method of Clause 15 or 16, further comprising a second membrane, wherein the second membrane extends over the first anchoring portion and the second anchoring portion without occluding the lumen when the interseptal occluding device is expanded.

Clause 18. The method of Clause 16 or 17, wherein the membrane comprises an elastic material.

Clause 19. The method of any of Clauses 16-18, wherein the membrane includes a plurality of perforations.

Clause 20. The method of any of Clauses 16-19, further comprising: leaving the interseptal occluding device within the patient's heart for a period, wherein the membrane is configured to promote tissue growth at least across the membrane.

Clause 21. The method of Clause 20, further comprising: inserting a medical instrument into the vasculature of the patient; advancing the medical instrument into the first compartment; and advancing the medical instrument through a tissue layer on the membrane and through the membrane, the medical instrument crossing the lumen into the second compartment.

Clause 22. The method of any of Clauses 16-21, wherein the membrane comprises a radiopaque material, wherein the radiopaque material identifies the lumen comprising a crossable region of the interseptal occluding device.

Clause 23. An interseptal occluding device configured to be implanted in a heart of a patient, the interseptal occluding device comprising: a support structure comprising a lumen; and a membrane comprising a plurality of electrospun fibers, the membrane coupled to at least a portion of the support structure, the membrane configured to occlude a majority of the lumen when the interseptal occluding device is implanted.

Clause 24. The interseptal occluding device of Clause 23, wherein the support structure further comprises a first anchoring portion and an opposite second anchoring portion, the lumen extending through a center of the first anchoring portion and a center second anchoring portion.

Clause 25. The interseptal occluding device of Clause 23 or 24, wherein the plurality of electrospun fibers have diameters between 0.5 microns and 5 microns.

Clause 26. The interseptal occluding device of any of Clauses 23-25, wherein the plurality of electrospun fibers are configured to deform with elongation of at least 400%.

Clause 27. The interseptal occluding device of any of Clauses 23-26, wherein the plurality of electrospun fibers have an ultimate strain between 350% and 600%.

Clause 28. The interseptal occluding device of any of Clauses 23-27, wherein the interseptal occluding device has an overall thickness of less than 5 mm.

Clause 29. The interseptal occluding device of any of Clauses 23-28, wherein, once implanted, the interseptal occluding device extends less than 0.1 mm into a left atrium of the heart.

Clause 30. The interseptal occluding device of any of Clauses 23-29 wherein, once implanted, the interseptal occluding device extends less than 1 mm into a right atrium of the heart.

Clause 31. The interseptal occluding device of any of Clauses 23-30, wherein the interseptal occluding device weighs less than 100 micrograms.

Clause 32. The interseptal occluding device of any of Clauses 23-31,wherein the plurality of electrospun fibers are configured to promote tissue growth at least across the membrane.

Clause 33. The interseptal occluding device of any of Clause 24-32, wherein the support structure is configured to contract and expand between a compressed tubular configuration for insertion through a patient's vasculature, and an expanded configuration, in which the first and second anchoring portions extend radially outwards from the lumen.

Clause 34. The interseptal occluding device of any of Clauses 23-33, wherein the membrane is less than 100 microns thick.

Clause 35. The interseptal occluding device of any of Clauses 23-34, wherein, once implanted, discontinuities of the interseptal occluding device extend less than 0.1 mm into the heart.

Clause 36. The interseptal occluding device of any of Clauses 23-35, wherein, the support structure is encapsulated within a second membrane.

Clause 37. An interseptal occluding device configured to be implanted in a heart of a patient, the interseptal occluding device comprising: a support structure comprising a central structure; and a membrane configured to promote tissue growth across the central structure, the membrane coupled to at least a portion of the support structure, the membrane configured to occlude a majority of the central structure when the interseptal occluding device is implanted.

Clause 38. The interseptal occluding device of Clause 37, wherein the support structure further comprises a first anchoring portion and an opposite second anchoring portion, the central structure extending through a center of the first anchoring portion and a center second anchoring portion.

Clause 39. The interseptal occluding device of Clause 37 or 38, wherein the membrane comprises a plurality of electrospun fibers.

Clause 40. The interseptal occluding device of Clause 39, wherein the plurality of electrospun fibers have diameters between 0.5 microns and 5 microns.

Clause 41. The interseptal occluding device of Clause 39 or 40, wherein the plurality of electrospun fibers are configured to deform with elongation of at least 400%.

Clause 42. The interseptal occluding device of any of Clauses 39-41, wherein the plurality of electrospun fibers have an ultimate strain between 350% and 600%.

Clause 43. The interseptal occluding device of any of Clauses 37-42, wherein the interseptal occluding device has an overall thickness of less than 4 mm.

Clause 44. The interseptal occluding device of any of Clauses 37-43,wherein, once implanted, the interseptal occluding device extends less than 0.1 mm into a left atrium of the heart.

Clause 45. The interseptal occluding device of any of Clauses 37-44 wherein, once implanted, the interseptal occluding device extends less than 1 mm into a right atrium of the heart.

Clause 46. The interseptal occluding device of any of Clauses 37-45, wherein the interseptal occluding device weighs less than 100 micrograms.

Clause 47. The interseptal occluding device of any of Clauses 39-46, wherein the plurality of electrospun fibers are configured to promote tissue growth at least across the membrane.

Clause 48. The interseptal occluding device of any of Clause 38-47, wherein the support structure is configured to contract and expand between a compressed tubular configuration for insertion through a patient's vasculature, and an expanded configuration, in which the first and second anchoring portions extend radially outwards from the central structure.

Clause 49. The interseptal occluding device of any of Clauses 37-48, wherein the membrane is less than 100 microns thick.

Clause 50. The interseptal occluding device of any of Clauses 37-49, wherein the support structure is encapsulated within a second membrane.

Clause 51. An interseptal occluding device comprising: a support structure comprising a central structure, a first anchoring portion and an opposite second anchoring portion; and a membrane coupled to the first anchoring portion, the membrane configured to occlude a majority of the central structure when the support structure is expanded, the membrane configured to promote tissue growth at least across the membrane.

Clause 52. An interseptal occluding device comprising: a support structure comprising: a first anchoring portion; a second anchoring portion opposite the first anchoring portion; and an expandable central structure coupled to the first anchoring portion and the second anchoring portion; a first membrane coupled to the first anchoring portion; and a second membrane coupled to the second anchoring portion.

Clause 53. The interseptal occluding device of Clause 52, wherein the expandable central structure comprises a resilient member.

Clause 54. The interseptal occluding device of Clause 52 or Clause 53, wherein the expandable central structure comprises a spring, the spring configured to bias the first anchoring portion towards the second anchoring portion.

Clause 55. The interseptal occluding device of any of Clauses 52-54, wherein the first membrane and the second membrane comprise an elastic material.

Clause 56. The interseptal occluding device of any of Clauses 52-55, wherein the first membrane and the second membrane include a plurality of perforations.

Clause 57. The interseptal occluding device of any of Clauses 52-56, wherein the first membrane and the second membrane comprise a radiopaque material.

Clause 58. The interseptal occluding device of any of Clauses 52-57, wherein the support structure is configured to contract and expand between a compressed tubular configuration for insertion through a patient's vasculature, and an expanded configuration, in which the first and second anchoring portions extend radially outwards from the expandable central structure.

Clause 59. The interseptal occluding device of any of Clauses 52-58, wherein the first and second anchoring portions comprise a shape-memory material.

Clause 60. The interseptal occluding device of any of Clauses 52-59, wherein at least one or the first anchoring portion and the second anchoring portion comprises a plurality of loops, wherein the plurality of loops is configured to releasably couple the interseptal occluding device to a delivery device.

Clause 61. The interseptal occluding device of any of Clauses 52-60, wherein the first anchoring portion and the second anchoring portion are configured to compress a partition of a heart therebetween.

Clause 62. The interseptal occluding device of Clause 61, wherein the first and second membranes are configured to allow a medical instrument inserted in a first compartment of the heart to pass through the first and second membranes and a tissue layer formed on the first and second membranes into a second compartment of the heart, wherein the partition divides the first and second compartments of the heart.

Clause 63. The interseptal occluding device of any of Clauses 52-62, wherein the first membrane forms a continuous surface, and the second membrane forms a continuous surface.

Clause 64. The interseptal occluding device of any of Clauses 52-63, wherein the first and second membranes prevent the support structure from directly contacting a patient's blood when the interseptal occluding device is implanted in the patient.

Clause 65. The interseptal occluding device of Clause 61, wherein a lumen extends through a center of the first anchoring portion, a center second anchoring portion, and a center of the expandable central structure, the lumen delimiting an opening in the partition.

Clause 66. The interseptal occluding device of any of Clauses 52-65, wherein the first and second membranes comprise a plurality of electrospun fibers.

Clause 67. The interseptal occluding device of Clause 66, wherein the plurality of electrospun fibers have diameters between 0.5 microns and 5 microns.

Clause 68. The interseptal occluding device of Clause 66 or Clause 67, wherein the plurality of electrospun fibers are configured to deform with elongation of at least 400%.

Clause 69. The interseptal occluding device of any of Clauses 66-68, wherein the plurality of electrospun fibers have an ultimate strain between 350% and 600%.

Clause 70. The interseptal occluding device of any of Clauses 66-69 ,wherein the plurality of electrospun fibers are configured to promote tissue growth at least across the first membrane and the second membrane.

Clause 71. The interseptal occluding device of any of Clauses 52-70, wherein the interseptal occluding device has an overall thickness of less than 5 mm.

Clause 72. The interseptal occluding device of any of Clauses 52-71, wherein, once implanted, the interseptal occluding device extends less than 1 mm into a right atrium of a heart.

Clause 73. The interseptal occluding device of any of Clauses 52-72, wherein, once implanted, the interseptal occluding device extends less than 1 mm into a left atrium of a heart.

Clause 74. The interseptal occluding device of any of Clauses 52-73, wherein the interseptal occluding device weighs less than 100 micrograms.

Clause 75. The interseptal occluding device of any of Clauses 52-74. Wherein the first membrane and the second membrane are less than 100 microns thick.

Clause 76. The interseptal occluding device of any of Clauses 52-75, wherein, once implanted, discontinuities of the interseptal occluding device extend less than 0.1 mm into a heart.

Clause 77. A delivery system for an implantable device, the delivery system comprising: a handle; a sheath comprising: a first end; a second end distal to the first end; and a channel extending between the first end and the second end, the sheath extending from the handle; a lumen comprising: a third end; and a fourth end distal to the third end, the lumen configured to travel within the channel; a delivery apparatus comprising: a connector body coupled to the lumen near the fourth end; and a plurality of implant grasping arms, each implant grasping arm including a distal arm tip, the plurality of implant grasping arms extending distally to the connector body, the plurality of implant grasping arms configured to releasably connect to an implantable device at the distal arm tips; and a lock tube assembly comprising: a plurality of lock tubes; and a lock tube holder, the plurality of lock tubes extending distally from the lock tube holder, the lock tube holder coupled to the fourth end; wherein each implant grasping arm extends through an individual lock tube of the plurality of lock tubes, the plurality of lock tubes configured to move between a locked configuration and an unlocked configuration, in the locked configuration, the plurality of lock tubes extending over the distal arm tips, in the unlocked configuration, the plurality of lock tubes moving proximately to expose the distal arm tips, wherein in the unlocked configuration, the implantable device is releasable from the delivery system.

Clause 78. The delivery system of Clause 77, wherein the handle includes an actuator configured to move the plurality of lock tubes between the locked configuration and the unlocked configuration.

Clause 79. The delivery system of Clause 78, wherein axial movement of the actuator causes axial movement of the plurality of lock tubes.

Clause 80. The delivery system of Clause 78, wherein rotational movement of the actuator causes axial movement of the plurality of lock tubes.

Clause 81. The delivery system of any of Clauses 77 to 80, further comprising a guidewire, the guidewire extending through a second channel of the lumen.

Clause 82. The delivery system of any of Clauses 77 to 81, wherein the implantable device moves from a compressed tubular configuration for travel through the sheath, and an expanded configuration upon exiting the second end of the sheath.

Clause 83. The delivery system of any of Clauses 77 to 82, wherein each distal arm tip comprises a flag with a cutout, wherein a portion of the implantable device is received within the cutouts prior to release of the implantable device, the plurality of lock tubes extending over the flags in the locked configuration.

Clause 84. The delivery system of any of Clauses 77 to 83, wherein the distal end of the lumen comprises an internally threaded cylinder and the lock tube holder includes an externally threaded shaft, the internally threaded cylinder positioned within the connector body and axially fixed relative to the connector body, the externally threaded shaft threadedly engaged with the internally threaded cylinder, wherein rotation of the lumen causes axial movement of the lock tube holder.

Clause 85. The delivery system of any of Clauses 77 to 84, wherein the plurality of implant grasping arms are configured to move between a compressed configuration for travel within the sheath and an expanded configuration in which the distal arm tips move outwardly from each other.

Clause 86. The delivery system of any of Clauses 77 to 85, wherein the implantable device comprises the interseptal occluding device of any of Clauses 1 to 13 or Clauses 23 to 76.

Clause 87. A method of implanting an occluding device within a partition opening of a heart, the method comprising: coupling a distal end of a delivery apparatus to outer tips of a first anchoring portion of the occluding device, the occluding device in a compressed configuration, the outer tips radially outward of a center of the occluding device in an expanded configuration; advancing the occluding device and the delivery apparatus through a sheath positioned across the partition opening; advancing the occluding device and the delivery apparatus until a second anchoring portion of the occluding device protrudes beyond the sheath and extends radially outwards into a second side of the partition opening; retracting the sheath into a first side of the partition opening; advancing the occluding device and the delivery apparatus until the first anchoring portion protrudes beyond the sheath and extends radially outwards into the first side of the partition opening; retracting a plurality of lock tubes of the delivery apparatus to expose the outer tips of the first anchoring portion; and releasing the occluding device from the delivery apparatus.

Clause 88. The method of Clause 87, further comprising: retracting the delivery apparatus and the occluding device into the sheath, wherein retracting the delivery apparatus causes the occluding device to move from the expanded configuration to the compressed configuration.

Clause 89. The method of Clause 88, further comprising: repositioning the sheath across the partition opening; advancing the occluding device and the delivery apparatus until the second anchoring portion of the occluding device protrudes beyond the sheath and extends radially outwards into the second side of the partition opening; retracting the sheath into the first side of the partition opening; and advancing the occluding device and the delivery apparatus until the first anchoring portion protrudes beyond the sheath and extends radially outwards into the first side of the partition opening.

Clause 90. The method of any of Clause 87 to 89, wherein the delivery apparatus is coupled to a lumen, the lumen configured to move the delivery apparatus and the occluding device proximally and distally within the sheath.

Clause 91. The method of Clause 90, wherein the delivery apparatus comprises: a connector body coupled to the lumen near a distal end of the lumen; and a plurality of implant grasping arms, each implant grasping arm including a distal arm tip, the plurality of implant grasping arms extending distally to the connector body, the plurality of implant grasping arms configured to releasably connect to outer tips of the first anchoring portion at the distal arm tips.

Clause 92. The method of Clause 91, wherein the delivery apparatus further comprises: a lock tube holder, the plurality of lock tubes extending distally from the lock tube holder, the lock tube holder coupled to the distal end of the lumen, wherein each implant grasping arm extends through an individual lock tube of the plurality of lock tubes, the plurality of lock tubes configured to move between a locked configuration and an unlocked configuration, in the locked configuration, the plurality of lock tubes extending over the distal arm tips, in the unlocked configuration, the plurality of lock tubes moving proximately to expose the distal arm tips, wherein in the unlocked configuration, the occluding device is releasable from the delivery apparatus.

Clause 93. The method of Clause 92, wherein each distal arm tip comprises a flag with a cutout, wherein the outer tips of the first anchoring portion are received within the cutouts prior to release of the occluding device, the plurality of lock tubes extending over the flags in the locked configuration.

Clause 94. The method of Clause 93, wherein the distal end of the lumen comprises an internally threaded cylinder and the lock tube holder includes an externally threaded shaft, the internally threaded cylinder positioned within the connector body and axially fixed relative to the connector body, the externally threaded shaft threadedly engaged with the internally threaded cylinder, wherein rotation of the lumen causes axial movement of the lock tube holder.

Clause 95. The method of any of Clauses 91 to 94, wherein the plurality of implant grasping arms are configured to move between a compressed configuration for travel within the sheath and an expanded configuration in which the distal arm tips move outwardly from each other.

Clause 96. The method of any of Clause 87 to 95, wherein the sheath extends from a handle, the handle including an actuator configured to move the plurality of lock tubes between the locked configuration and the unlocked configuration.

Clause 97. The method of Clause 96, wherein axial movement of the actuator causes axial movement of the plurality of lock tubes.

Clause 98. The method of Clause 96, wherein rotational movement of the actuator causes axial movement of the plurality of lock tubes.

Clause 99. The method of any of Clause 90 to 98, wherein the lumen is advanced over a guidewire, the guidewire extending through the lumen, the occluding device, and the delivery apparatus.

Clause 100. The method of any of Clauses 87 to 99, wherein the occluding device comprises the interseptal occluding device of any of Clauses 1 to 13 or Clauses 23 to 76.

1-100. (canceled)

101. An interseptal occluding device configured to be positioned within an opening in a partition of a heart, the interseptal occluding device comprising:
a support structure comprising:
a first anchoring portion;
a second anchoring portion opposite the first anchoring portion; and
a lumen extending through a center of the first anchoring portion and a center of the second anchoring portion, the lumen being unobstructed by the first anchoring portion and the second anchoring portion,
wherein the support structure is configured to contract and expand between a compressed tubular configuration for insertion through a patient's vasculature, and an expanded configuration, in which the first anchoring portion and the second anchoring portion extend radially outwards from the lumen to compress the partition therebetween, with the lumen extending through the opening in the partition of the heart; and
a membrane comprising a plurality of electrospun fibers, the membrane coupled to the first anchoring portion, the membrane configured to occlude the lumen when the support structure is expanded to prevent blood flow from passing through the opening in the partition of the heart, the membrane configured to promote tissue growth at least across the membrane.

102. The interseptal occluding device of claim 101, wherein the membrane includes a plurality of perforations.

103. The interseptal occluding device of claim 101, wherein the membrane comprises a radiopaque material.

104. The interseptal occluding device of claim 101, further comprising a second membrane, wherein the second membrane extends over the first anchoring portion and the second anchoring portion without occluding the lumen when the support structure is expanded.

105. The interseptal occluding device of claim 104, wherein the second membrane prevents the support structure from directly contacting a patient's blood when the interseptal occluding device is implanted in the patient.

106. The interseptal occluding device of claim 101, wherein the second anchoring portion comprises a plurality of loops located radially outward of the lumen and at or near an outer edge of the second anchoring portion, wherein the plurality of loops are configured to releasably couple the interseptal occluding device to a delivery device.

107. The interseptal occluding device of claim 101, wherein the membrane is configured to allow a medical instrument inserted in a first compartment of the heart to pass through the membrane and a tissue layer formed on the membrane through the lumen into a second compartment of the heart, wherein the partition divides the first compartment and the second compartment.

108. The interseptal occluding device of claim 101, wherein the membrane forms a continuous surface.

109. The interseptal occluding device of claim 101, wherein the membrane is configured to lie flat against the partition once implanted.

110. An interseptal occluding device configured to be positioned within an opening in a partition of a heart, the interseptal occluding device comprising:
a support structure comprising a lumen; and
a membrane comprising a plurality of electrospun fibers, the membrane coupled to at least a portion of the support structure, the membrane configured to occlude a majority of the lumen when the interseptal occluding device is implanted,
wherein the interseptal occluding device extends less than 0.1 mm into a left atrium of the heart once implanted.

111. The interseptal occluding device of claim 110, wherein the plurality of electrospun fibers have diameters between 0.5 microns and 5 microns.

112. The interseptal occluding device of claim 110, wherein the plurality of electrospun fibers are configured to deform with elongation of at least 400%.

113. The interseptal occluding device of claim 110, wherein the plurality of electrospun fibers have an ultimate strain between 350% and 600%.

114. The interseptal occluding device of claim 110, wherein the interseptal occluding device has an overall thickness of less than 5 mm.

115. The interseptal occluding device of claim 110, wherein, once implanted, the interseptal occluding device extends less than 1 mm into a right atrium of the heart.

116. The interseptal occluding device of claim 110, wherein the interseptal occluding device weighs less than 200 micrograms.

117. The interseptal occluding device of claim 110, wherein the plurality of electrospun fibers are configured to promote tissue growth at least across the membrane.

118. The interseptal occluding device of claim 110, wherein the membrane is less than 100 microns thick.

119. The interseptal occluding device of claim 110, wherein the support structure comprises:
a first anchoring portion;
a second anchoring portion opposite the first anchoring portion; and
an expandable central structure coupled to the first anchoring portion and the second anchoring portion, the expandable central structure defining the lumen.

120. The interseptal occluding device of claim 119, wherein the expandable central structure comprises a resilient member.
